(19) United States
(12) Patent Application Publication
(10) Pub. No.: US 2024/0398950 A1
(43) Pub. Date: Dec. 5, 2024

(54) ENHANCING ADOPTIVE CELL TRANSFER BY PROMOTING A SUPERIOR POPULATION OF ADAPTIVE IMMUNE CELLS

(71) Applicant: cellvie Inc., Houston, TX (US)

(72) Inventor: Nina DUMAUTHIOZ, Winterthur (CH)

(21) Appl. No.: 18/699,134

(22) PCT Filed: Oct. 6, 2022
(86) PCT No.: PCT/US22/77721

§ 371 Date: Apr. 5, 2024

Related U.S. Application Data
(60) Provisional application No. 63/253,058, filed on Oct. 6, 2021.

(51) Int. Cl. A61K 39/00; A61P 35/00; C07K 14/725; C07K 16/28; C12N 15/88

(52) U.S. Cl. CPC A61K 39/464412; A61K 39/4611; A61K 39/4631; A61P 35/00; C07K 14/7051; C07K 16/2803; C12N 15/88

(57) ABSTRACT

The disclosure relates to mitochondria-enhanced immune cells, their compositions and therapeutic use.

### CROSS REFERENCE TO RELATED APPLICATIONS

This application claims the benefit of U.S. Provisional Application No. 63/253,058 filed Oct. 6, 2021, which is hereby incorporated in its entirety by reference for all purposes.

### FIELD

The invention relates to the field of biomedicine and specifically methods useful for therapy of cancer, infectious and autoimmune diseases. In particular, the present invention is directed to a therapeutic treatment using mitochondria-enhanced immune cells, such as but not limited to, mitochondria-enhanced adaptive immune cells. The present invention is directed to mitochondria-enhanced immune cells for use in treating cancer, infectious and autoimmune diseases. In particular, the immune cells of the present invention are immune cells, such as but not limited to, T immune cells or propagated in vitro T cells. More in particular, the immune cells of the present invention are immune cells, such as but not limited to, CD8 immune cells circulating in the blood, tumor-infiltrating lymphocytes (TILs), engineered T cells, chimeric antigen receptor (CAR) T-cells, CD4 immune cells circulating in the blood, immunosuppressive regulatory T cells (Treg cells), effector T cells, memory T cells, alpha-beta T cells (αβ T cells), and gamma-delta T cells (γδ T cells). The mitochondria-enhanced immune cells have an improved persistence, higher survival, and/or differentiation capacity. More in particular, the present invention relates to mitochondria-enhanced memory T cell (e.g., memory T cell transplanted with exogenous mitochondria), which have improved persistence, higher survival capacity and/or higher differentiation capacity. The mitochondria-enhanced memory T cell of the invention demonstrate an increased efficacy in the adoptive cell transfer therapy (ACT) due to the enhancement, for instance, of the proportion of highly persistent cells in the bulk population. The present invention further provides mitochondria-enhanced immunosuppressive regulatory T cells (Treg) (e.g., Treg CD4 T cells transplanted with exogenous mitochondria), which have improved survival capacity and/or higher differentiation capacity for the treatment of autoimmune diseases and transplanted organ or tissue rejection. The present invention is directed to pharmaceutical compositions comprising mitochondria-enhanced immune cells. The present invention relates to increasing efficacy of cellular technologies leading to generation of higher proportions of the immune T cells or higher proportion of the immune T cells at certain stage of differentiation, which have improved persistence, higher survival capacity and/or higher differentiation capacity.

### BACKGROUND

Cancer and autoimmunity share a common origin but exert powerful forces that work in opposite directions. Both diseases result from failures in the body's immune system. Cancer often develops because the immune system failed to do its job in recognizing and/or attacking defective and/or transformed cells, allowing the cells to divide and grow. Conversely, an autoimmunity—a faulty immune response that leads to diseases such as colitis and lupus—occurs when the immune system has mistakenly attacked healthy cells. Almost any part of the body can be targeted by the immune system, including the heart, brain, nerves, muscles, connective tissues, skin, eyes, lungs, kidneys, the digestive tract, blood cells and blood vessels.

Cancer is one of the leading causes of death in the developed world, with an estimated 1.9 million new cancer cases diagnosed and 608,570 cancer deaths in the United States in 2021 (https://www.cancer.org/content/dam/cancer-org/research/cancer-facts-and-statistics/annual-cancer-facts-and-figures/2021/cancer-facts-and-figures-2021.pdf). According to the World Health Organization (WHO), cancer is a leading cause of death worldwide, accounting for nearly 10 million deaths in 2020 (Ferlay J, Ervik M, Lam F, Colombet M, Mery L, Pifferos M, et al. Global Cancer Observatory: Cancer Today. Lyon: International Agency for Research on Cancer; 2020 (https://gco.iarc.fr/today, accessed February 2021). The most common in 2020 (in terms of new cases of cancer) were: breast (2.26 million cases); lung (2.21 million cases); colon and rectum (1.93 million cases); prostate (1.41 million cases); skin (non-melanoma) (1.20 million cases); and stomach (1.09 million cases). The most common causes of cancer death in 2020 were: lung (1.80 million deaths); colon and rectum (935′000 deaths); liver (830′000 deaths); stomach (769′000 deaths); and breast (685′000 deaths).

Cancer results from defective cells, which acquired mutations, allowing them to bypass the normal cell cycle checkpoints. Over time, the excessive growth of mutated cells creates a heterogenous tumor mass composed of various cell types. Upon acquisition of hallmark of metastasis, such as motility and invasion capacities, abilities to modulate the environment to favor cancer cell survival, cancer cells can disseminate in the body, creating distant metastasis away from the original tumor bed.

Immune cells are crucial players to detect, control and eradicate cancer cells and pathogens. T cell receptors (TCRs) on the surface of T lymphocytes recognize antigenic peptide fragments presented on Major histocompatibility complex (MHC) molecules. During an acute infection, naïve T cells specific against the invading pathogen are activated through their TCR in the context of MHC/antigen presentation, clonally expanded, and give rise to effector cells. Through direct killing, effector cells mediate the removal of infected cells from the body. Upon pathogen clearance, the mounted immune response contracts, leading to apoptosis of the majority of the activated CD8 T cells. A part of the antigen-specific T cells further differentiates to generate the memory T cell pool providing a long-term protection to the individual (FIG. 1). Of note, memory cells have distinct hallmarks, such as enhanced persistence, self-renewal ability and efficient recall capacity upon re-infection with the encountered pathogen. In case of a second infection with the identical pathogen, the triggered mounted immune response by memory cells will occur faster and stronger compared to naïve T cells (Vanja Lazarevic et al., “T-bet: a bridge between innate and adaptive immunity” Nat Rev Immunol. 2013 November; 13(11): 777-789).

Interestingly, metabolism of naïve, effector and memory CD8 T cells has been shown to differ. Naïve CD8 T cells are quiescent, relying mostly on oxidative phosphorylation (OXPHOS) to supply their energy demands. Upon activation, effector CD8 T cells favor glycolysis to sustain their effector functions and clonal expansion. Indeed, the breaking down of glucose molecules takes part in the generation of crucial building blocks to meet the requirements of their high proliferation. On the other hand, memory CD8 T cells rely on OXPHOS and fatty acid oxidation (FAO). It was shown that memory cells have more mitochondrial mass than naïve T cells. The reliance of memory cells on mitochondria to sustain their ATP production give them a bioenergetic advantage. Indeed, memory CD8 T cells display an enhanced respiratory reserve, measured as the spare respiratory capacity (SRC) (Gerritje J. W. van der Windt et al.; Immunity, 2012 January 27; 36(1): 68-78; Guillermo O. Rangel Rivera et al., Front. Immunol., 18 Mar. 2021|https://doi.org/10.3389/fimmu.2021.645242).

Different subsets of memory CD8 T cells have complementary roles or localization. Amongst other: stem cell-like memory, effector memory, central memory and tissue-resident memory can be highlighted. Both stem cell-like memory and central memory T cells express CD62L, a L-selectin mediating adhesion and allowing the homing to secondary lymphoid tissues. This unique ability to enter the lymph nodes (LN) leads to the optimized screening of antigen presenting cells (APC) harboring various antigens at their surface and strong recall responses induced by central memory cells. Effector memory T cells have a restricted circulation to the bloodstream and display direct cytotoxic and effector functions upon re-infection with the encountered pathogen. Conversely, tissue-resident memory cells do not circulate, and instead localize in peripheral tissue such as skin, lung and gut to efficiently block pathogen at diverse entry sites.

The immune system is educated to tolerate and not react against self, consequently, cancer cells may not be detected with the same intensity than an invading pathogen. In cancer patients, T cells normally build poor or no response against syngeneic transformed cells, (i) because of their poor antigenicity, (ii) for the transformed cells are not phenotypically foreign, and (iii) due to the generalized immunosuppressive conditions often associated with cancer (Medler et al., 2015, “Immune response to cancer therapy: mounting an effective antitumor response and mechanisms of resistance”, Trends Cancer 1:66-75).

Interestingly, targeting certain immunosuppressive mechanisms by checkpoint blockade therapy enhances the mounted immune response against cancer. In addition, there may be a metabolic competition at the tumor site between cancer cells and the infiltrating immune cells. The high reliance of cancer cells on glycolysis is often observed, leading to reduce glucose, one fuel source, from the intra-tumoral environment. Inability to engage glycolysis in effector CD8 T cells has a drastic negative impact on their effector functions and killing capacity.

T cell-based immunotherapy uses the immune system of the cancer patient to target its own tumor mass. The immune cells are extracted directly from the tumor, such as tumor infiltrating lymphocytes (TILs) or from the blood, such as peripheral blood mononuclear cells (PBMC). TILs with the correct antitumor specificity can be selected by cell culture methods and validated killing capacity. CD8 T cells extracted from the blood can be modified to acquire a tumor reactivity, such as through a chimeric antigen receptor (CAR). TILs or CAR-T cells are cultivated in vitro such as in presence of high dose of IL-2 promoting a strong expansion before being re-infused to the patient, a method referred as adoptive cell transfer (ACT) (Rohaan, M. W., Wilgenhof, S. & Haanen, J. B. A. G., “Adoptive cellular therapies: the current landscape”, Virchows Arch 474, 449-461 (2019). One advantage of ACT lies within its high specificity compared to conventional therapies, such as chemotherapy, radiation therapy and surgery. In addition, in some cancers, such as melanoma and lung cancer, ACT with autologous TILs represents the most efficient way to treat patients. Advanced melanoma patients treated with conventional chemotherapy show an overall survival of 10%, whereas post ACT, the overall survival increases to 41% (Larkin, James et al. “Overall Survival in Patients With Advanced Melanoma Who Received Nivolumab Versus Investigator's Choice Chemotherapy in CheckMate 037: A Randomized, Controlled, Open-Label Phase III Trial.” Journal of clinical oncology: official journal of the American Society of Clinical Oncology vol. 36, 4 (2018): 383-390. doi:10.1200/JCO.2016.71.8023; Dafni, U et al. “Efficacy of adoptive therapy with tumor-infiltrating lymphocytes and recombinant interleukin-2 in advanced cutaneous melanoma: a systematic review and meta-analysis.” Annals of oncology: official journal of the European Society for Medical Oncology vol. 30, 12 (2019): 1902-1913. doi:10.1093/annone/mdz398). Along the same line, CAR therapy targeting CD19 expression has demonstrated consistently high antitumor efficacy in children and adults affected by relapsed B-cell acute lymphoblastic leukemia (B-ALL), chronic lymphocytic leukemia (B-CLL), and non-Hodgkin lymphoma (NHL), with percentage of complete remissions ranging from 70 to 94% in different clinical trials (Wang et al., 2017, “New development in CAR-T cell therapy”, J Hematol Oncol 10:53; Morotti, M., Albukhari, A., Alsaadi, A. et al., “Promises and challenges of adoptive T-cell therapies for solid tumours”, Br J Cancer 124, 1759-1776 (2021)). ACT has yet to realize its potential for treating a wide variety of diseases including cancer, infectious disease, autoimmune disease, inflammatory disease, and immunodeficiency. Nevertheless, hurdles remain to be overcome regarding ACT therapy. Patients that display no or too low amount of tumor infiltrating T cells will not benefit from this therapy. Tumors can be classified as “hot” tumors, i.e., tumors with elevated levels of T cells infiltration and “cold” tumors, i.e., tumors with low levels of T cells infiltration. “hot” tumors are to be preferred for the collection of a sufficient amount of CD8 T cells for in vitro expansion before re-infusion. Moreover, cultured TILs should maintain effector functions, proliferation capacity and self-renewal ability to induce a strong antitumor response upon ACT. Consequently, the infusion of terminally differentiated TILs, such as cells with limited proliferation and self-renewal capacity is deleterious regarding the clinical outcome of the patient. Importantly, the finest selection of TILs which display memory-like phenotype will improve the antitumor response post transfer to the patient. Current methods to target one subset within the extracted TILs is time consuming, may metabolically challenge the cells and might require surface fluorescent labelling such as sorting by flow cytometry.

Despite, demonstrating high potency against hematological malignancies, strong antitumor response elicited by CAR-T cells is often associated with toxicity (i.e., severe cytokine-release syndrome and neurotoxicity), while patients with poor CAR-T proliferation and persistence show reduced rates of durable remissions. Taking together, the selection of memory-like TILs or CAR-T cells in a simple and efficient manner, will be drastically beneficial to the clinical outcome, by potentially improving the duration of the mediated response.

Autoimmunity—a faulty immune response that leads to diseases such as colitis and lupus—occurs when the immune system has mistakenly attacked healthy cells. Almost any part of the body can be targeted by the immune system, including the heart, brain, nerves, muscles, connective tissues, skin, eyes, lungs, kidneys, the digestive tract blood cells and blood vessels. A broad range of autoimmune diseases exist given that they vary according to the part of the body that is being targeted by the immune system. Common autoimmune diseases include rheumatoid arthritis, systemic lupus erythematosus, multiple sclerosis, autoimmune vasculitis, myasthenia gravis, pernicious anemia, Hashimoto's thyroiditis, type 1 diabetes, inflammatory bowel disease (IBS), Addison's disease, Grave's disease, Sjögren's syndrome, psoriasis, and celiac diseases. To date, the American Autoimmune Related Disease Association (AARDA) has classified more than 100 autoimmune diseases, making it the third most common type of disease in the United States. In fact, autoimmune diseases affect 5 to 10% of the global population, particularly women, who are two to ten times more likely to suffer from an autoimmune disease than men. Although most diseases can occur at any age, some diseases primarily occur in childhood and adolescence (e.g. type 1 diabetes), in the mid-adult years (e.g. myasthenia gravis, multiple sclerosis), or among older adults (e.g. rheumatoid arthritis, primary systemic vasculitis) (Wang et al., 2015, “Human autoimmune diseases: a comprehensive update”, J Intern Med 278:369-95).

Regulatory T cells (Treg) belong to the CD4 T cell compartment and are crucial players to control, reduce or treat autoimmune diseases. They rely on mitochondria to sustain their functions and energetical needs. Treg work to balance the mounted immune response to allow an appropriate response against an invading pathogen and avoid or limit tissue damages targeted by the immune system. They mediate their role to dampen the immune response (i) by direct binding to immune cells, (ii) by producing anti-inflammatory cytokines, such as IL-10 and IL-35 and (iii) by competing for the survival signal IL-2 with a higher affinity. Polyclonal Treg therapy uses the same principle as ACT, consequently extracted autologous Treg are expanded in vitro before being re-infused to the patient aiming to restore the balance of a mounted immune response (Peter J. Eggenhuizen et al., “Treg Enhancing Therapies to Treat Autoimmune Diseases”, Int J Mol Sci. 2020 October; 21(19): 7015).

Thus, the technical problem underlying the present invention is to provide new therapeutics and therapeutic strategies for selection of persistent or memory-like TILs and CD8 T cells from the blood or Treg from the CD4 compartment. The present invention aims to increase the proportion post in vitro expansion of CD8 T cells with higher survival capacity to be used in the context of ACT or Treg to be selected for Treg therapy. The solution of said technical problem is achieved by providing the embodiments characterized in the claims.

### SUMMARY

The present disclosure relates to mitochondria-enhanced immune cells, their compositions and therapeutic use.

The present disclosure provides immune cells, e.g. human immune cells, treated with isolated viable mitochondria or comprising exogenous isolated viable mitochondria in an amount effective to enhance the survival and/or to promote the selection of adaptive immune cells, e.g. human adaptive immune cells, such as B cells or T cells, preferably T cells, such as CD4 immune T cells or CD8 immune T cells, relative to adaptive immune cells, e.g. human adaptive immune cells, not treated with isolated viable mitochondria or not comprising exogenous viable mitochondria. In some aspects the mitochondria of the present disclosure enhance the survival and/or to promote the selection of memory CD8 T cells, such as central memory CD8 T cells and effector memory CD8 T cells. In some other aspects, the viable mitochondria are in an amount effective to enhance the survival and/or to promote the selection of regulatory T (Treg) cells, such as Treg CD4 cells.

Provided for herein is a composition comprising immune cells or a composition of immune cells treated with isolated viable mitochondria or comprising exogenous isolated viable mitochondria. The composition can further comprise one or more pharmaceutically acceptable carriers.

Also provided for herein is a method of enhancing the survival and/or promoting the selection of immune cells or a population of immune cells, such as adaptive immune cells, e.g. human T cells, comprising the step of: (a) activating the immune cells in vitro in a cell-free medium with specific activating receptor agonist antibodies capable of driving the adaptive cells (such as T cells) activation; (b) exposing the immune cells to a pharmaceutical composition comprising isolated viable mitochondria for at least 3 days. In some aspects, the method of enhancing the survival and/or promoting the selection of immune cells or a population of immune cells comprises alternatively the step (a) activating the immune cells in vitro in a cell-free medium with coated CD3/CD28 beads, optionally in presence of recombinant interleukins, such IL-2; (b) exposing the immune cells to a pharmaceutical composition comprising isolated viable mitochondria for at least 3 days.

Also provided for herein are immune cells, e.g. human immune cells, such as human T cells, or a population of immune cells, treated with isolated viable mitochondria or comprising exogenous isolated viable mitochondria for use in a method of treating a subject in need thereof.

### BRIEF DESCRIPTION OF THE DRAWINGS

FIG. 1 Mounted immune response upon an acute infection.

FIG. 2 Schematic diagram of one exemplary protocol for isolating mitochondria from tissue or cultured cells.

FIG. 3A Increased proportion of central and effector memory CD8⁺ T cells day 9 post mitochondria transplantation. Fold change proportion of central memory CD8⁺ T cells upon mitochondria transplantation. CD8⁺ T cells from a bulk population were transplanted with exogenous mitochondria at dosage levels of 30 μg and 100 μg of mitochondria, as measured using a Qubit™ Protein Assay, per 1 million of CD8⁺ T cells day 12 post activation. On day 9 post transplantation, CD8⁺ T cells were stained, analyzed by flow cytometry using a FACSLyric (BD Biosciences) and classified as central memory (CD62L+, CD45RA−, CD45RO+) and effector memory (CD62L−, CD45RA−, CD45RO+).

Data are representative of three independent experiments presented as the mean±SD of three donors. *p<0.05; **p<0.01; ***p<0.001; ****p<0.0001.

FIG. 3B Increased proportion of central and effector memory CD8⁺ T cells day 9 post mitochondria transplantation. Fold change proportion of effector memory CD8⁺ T cells upon mitochondria transplantation. CD8⁺ T cells from a bulk population were transplanted with exogenous mitochondria at dosage levels of 30 μg and 100 μg of mitochondria, as measured using a Qubit™ Protein Assay, per 1 million of CD8⁺ T cells day 12 post activation. On day 9 post transplantation, CD8⁺ T cells were stained, analyzed by flow cytometry using a FACSLyric (BD Biosciences) and classified as central memory (CD62L+, CD45RA−, CD45RO+) and effector memory (CD62L−, CD45RA−, CD45RO+).

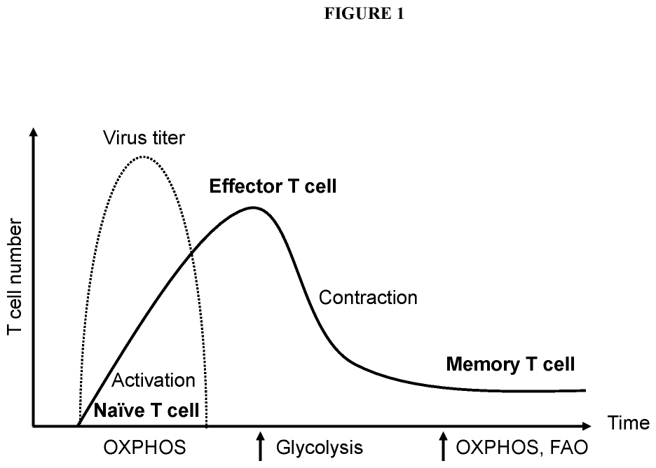

Data are representative of three independent experiments presented as the mean±SD of three donors. *p<0.05; **p<0.01; ***p<0.001; ****p<0.0001.

### DETAILED DESCRIPTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s) f one standard deviation of that value(s).

The term “isolated” means altered or removed from the natural state or environment. For example, a nucleic acid or a peptide naturally present in a living animal or cell is not “isolated,” but the same nucleic acid or peptide partially or completely separated from the coexisting materials of its natural state is “isolated.”

The term “mitochondria” or “mitochondrion” to be used herein refers to viable mitochondria that are (essentially) free of eukaryotic cell material, such as extraneous eukaryotic cell material, e.g. which have been isolated/purified from cells or a cell culture. Thus, only minimal amounts of cellular components (other than mitochondria) are present in (a composition of) mitochondria to be used herein. Preferably, no other cellular components than mitochondria are present in (a composition of) mitochondria to be used herein. Isolated mitochondria preferably exist in a substantially purified form, e.g. partially or completely separated from the coexisting materials of its natural state. In this sense, the “mitochondria” to be used herein are “isolated mitochondria” and the terms “mitochondria” and “isolated mitochondria” can be used interchangeably. Any current art-known technique may be used for isolation of mitochondria, such as for example, subcellular fractioning by repeated differential centrifugation (DC) or density gradient centrifugation (DGC). Accordingly, a mitochondrion of the present invention is preferably alive or viable and possesses a negative membrane potential. In the sense of the present invention “being alive” means having or maintaining a metabolism or another biological function or structure.

As used herein, the term “viable mitochondria” is used herein to describe viable mitochondria, which are intact, active, functioning and respiration-competent mitochondria. According to some embodiments, “viable mitochondria” refers to mitochondria that exhibit biological functions, such as, for example, respiration as well as ATP and/or protein synthesis.

As used herein, the term “intact mitochondria” is used throughout the specification to describe mitochondria, which comprise an integer outer and inner membrane, an integer inter-membrane space, integer cristae (formed by the inner membrane) and an integer matrix. Alternatively, intact mitochondria are mitochondria which preserve their structure and ultrastructure. In another aspect, intact mitochondria contain active respiratory chain complexes I-V embedded in the inner membrane, maintain membrane potential and capability to synthesize ATP.

As used herein, the term “transplantation” is used throughout the specification as a general term to describe the process of implanting an organ, tissue, mass of cells, individual cells, or cell organelles into a recipient. The term “cell transplantation” is used throughout the specification as a general term to describe the process of transferring at least one cell, e.g., an enhanced immune cell described herein, to a recipient. The terms include all categories of transplants known in the art, including blood transfusions. Transplants are categorized by site and genetic relationship between donor and recipient. The term includes, e.g., autotransplantation (removal and transfer of cells or tissue from one location on a patient to the same or another location on the same subject), allotransplantation (transplantation between members of the same species), and xenotransplantation (transplantations between members of different species).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “antibody” is used herein in its broadest sense and includes certain types of immunoglobulin molecules comprising one or more antigen-binding domains that specifically bind to an antigen or epitope. The term also includes non-immunoglobulin antigen-binding protein molecules, so-called antibody mimetics. An antibody specifically includes intact antibodies (e.g., intact immunoglobulins G, IgG), antibody fragments (e.g., Fab fragment, single-chain Fv (scFv), single domain antibodies, V_{H}, V_{L}, V_{HH}, NAR, tandem scFvs, diabodies, single-chain diabodies, DARTs, tandAbs, minibodies, single-domain antibodies (e.g., camelid V_{HH}), other antibody fragments or formats known to those skilled in the art), and antibody mimetics (e.g., adnectins, affibodies, affilins, anticalins, avimers, DARPins, knottins, etc.). The antibodies can be monospecific, bi- and multi-specific.

The term “antigen-binding domain” means the portion of an antibody or T cell receptor that is capable of specifically binding to an antigen or epitope via a variable domain. As used herein, “variable domain” refers to a variable nucleotide sequence that arises from a recombination event, for example, it can include a V, J, and/or D region of a T cell receptor (TCR) sequence from a T cell, such as an activated T cell, or it can include a V, J, and/or D region of an antibody. The term “antigen-binding fragment” refers to at least one portion of an antibody or TCR, or recombinant variants thereof, that contain the antigen binding domain, i.e., variable domains and hypervariable loops, so-called complementarity determining regions (CDRs), that are sufficient to confer recognition and specific binding of the antigen-binding fragment to a target, such as an antigen and its defined epitope. Examples of antigen-binding fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains (nanobodies), multi-specific antibodies generated from antibody fragments, and TCR fragments. Exemplary antibody and antibody fragment formats are described in detail in Brinkmann et al. (MABS, 2017, Vol. 9, No. 2, 182-212), herein incorporated by reference for all that it teaches.

The term “scFv” refers to a fusion protein comprising a variable fragment of the antibody heavy chain (V_{H}) linked in its C-terminus with an N-terminus of a variable fragment of the antibody light chain (V_{L}) via a flexible peptide linker, and capable of being expressed as a single polypeptide chain, and wherein the scFv retains the specificity of the intact antibody from which it is derived.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₃ or (Gly₄Ser)₄. In another embodiment, the linkers include multiple repeats of (Gly₂Ser), (GlySer) or (Gly₃Ser). Also included within the scope of the invention are linkers described in WO2012/138475 (incorporated herein by a reference).

“Heavy chain variable region” or “V_{H}” (or, in the case of the camelid single domain antibodies, e.g., nanobodies, “V_{HH}”) with regard to an antibody refers to the fragment of the heavy chain that contains three CDRs interposed between flanking stretches known as framework regions (FR); these framework regions are generally more conserved than the CDRs and form a scaffold to support the CDRs.

Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The term “antibody heavy chain,” refers to the larger of the two types of polypeptide chains present in an antibody molecule in their naturally occurring conformations, and which typically determines the immunoglobulin class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

The term “recombinant antibody” refers to an antibody that is generated using recombinant DNA technology, such as, for example, an antibody expressed by a bacterial, yeast, plant or mammalian cell. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “ag” refers to a molecule foreign to the body, such as present on a pathogen, that can be bound by an antibody or a T cell receptor. The antigen can be presented by antigen-presenting cells (APC) and under circumstances can trigger an immune response.

A person skilled in the art will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated by a chemical synthesis; it can also be derived from a biological sample, or might be a macromolecule besides a polypeptide, e.g., lipid or carbohydrate. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “immunosuppressive effect” refers to a biological effects which can inhibit or interfere with normal immune function.

A “human antibody” or “human TCR” is one which possesses an amino acid sequence corresponding to that of an antibody produced by a human or a human cell, or derived from a non-human source that utilizes a human antibody or TCR repertoire or human antibody/TCR-encoding sequences (e.g., obtained from human sources or designed de novo). Human antibodies and TCRs specifically exclude humanized antibodies and TCRs, respectively.

With regard to the binding of an antibody, TCR, or antigen-binding fragment thereof to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody, TCR, or antigen-binding fragment thereof to the target molecule is competitively inhibited by the control molecule. Specific binding, as used herein, can refer to an affinity in which the K_{D} value is below 10⁻⁶M, 10⁻⁷M, 10⁻⁸, 10⁻⁹, or 10⁻¹⁰M. Affinity can be measured by common methods known in the art, including those described herein, such as surface plasmon resonance (SPR) technology (e.g., BIACORE®) or biolayer interferometry (e.g., FORTEBIO®).

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “chimeric antigen receptor” or “CAR” refers to a recombinant polypeptide derived from the various polypeptides comprising an antigen-binding moiety (e.g., a polypeptide having at least an antigen-binding domain or antigen-binding fragment thereof) fused to a primary cytoplasmic signaling sequence (also referred to as a “primary signaling domain”) that acts in a stimulatory manner and that may contain a signaling motif which is known as immunoreceptor tyrosine-based activation motif or “ITAM”. Examples of an ITAM containing primary cytoplasmic signaling sequence that is of particular use in the invention includes, but is not limited to, those derived from CD3ζ (zeta), FcRγ (gamma), FcRβ (beta), CD3γ, CD3δ (delta), CD3ε (epsilon), CD5, CD22, CD79a, CD79b, CD278 (also known as “ICOS”) and CD66d. A CAR provides typically provides an engineered immune cell, such as a T lymphocyte, with antibody-type specificity or TCR-type specificity and activates some or all the functions of an effector cell, including the production of IL-2 and lysis of the target cells following signaling in T cells.

The antigen-binding domain or antigen-binding fragment thereof of the CARs described herein may exist in a variety of forms, for example where the antigen-binding domain is expressed as part of a contiguous polypeptide chain including, for example, a single domain antibody fragment (sdAb) or heavy chain antibody (HCAb), a single-chain Fv antibody (scFv), either naturally-derived, or synthetic, which binds to an antigen. The antigen-binding domain or antigen-binding fragment thereof of the CARs described herein can include any of the antibody formats or antibody fragment formats described herein. The antigen-binding domain or antigen-binding fragment thereof of the CARs described herein can include sequences that are not derived from antibodies, including but not limited to chimeric or artificial T-cell receptors (TCR). These chimeric/artificial TCRs may comprise a polypeptide sequence that recognizes a target antigen, where the recognition sequence may be, for example, but not limited to, the recognition sequence derived from a TCR or an scFv. The intracellular domain polypeptides are those that act to activate the T cell. Chimeric/artificial TCRs are discussed in, for example, Gross, G., and Eshhar, Z., FASEB Journal 6:3370-3378 (1992), and Zhang, Y., et al., PLOS Pathogens 6: 1-13 (2010).

A “CAR-T cell” is a T cell that has been transduced according to the methods disclosed herein and that expresses a CAR gene, e.g., incorporated randomly into the genome or purposely integrated into the CCR5 and AAVS1 loci, or into the T-cell receptor a constant (TRAC) locus. In some embodiments, the T cell is a CD4⁺ T cell, a CD8⁺ T cell, or a CD4⁺/CD8⁺ T cell. In some embodiments, the T cell is a regulatory T cell. In some embodiments, the T cell is autologous, allogeneic, or xenogeneic with reference to a subject.

As used herein, the term “subject” means a mammalian subject. The term “subject” is intended to include living organisms (e.g., mammals, human) in which an immune response can be elicited. Exemplary subjects include humans, monkeys, dogs, cats, mice, rats, cows, horses, camels, goats, rabbits, and sheep. In certain embodiments, the subject is a human. A “patient” is a subject suffering from or at risk of developing a disease, disorder or condition or otherwise in need of the compositions and methods provided herein.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

As used herein, the term “CD19”, B-lymphocyte antigen CD19, CD19 molecule (Cluster of Differentiation 19), B-Lymphocyte Surface Antigen B4, T-Cell Surface Antigen Leu-12 and CVID3 is a transmembrane protein that in humans is encoded by the gene CD19. In humans, CD19 is expressed in all B lineage cells, except for plasma cells, and in follicular dendritic cells. CD19 plays two major roles in human B cells. It acts as an adaptor protein to recruit cytoplasmic signaling proteins to the membrane and it works within the CD19/CD21 complex to decrease the threshold for B cell receptor signaling pathways. Due to its presence on all B cells, it is a biomarker for B lymphocyte development, lymphoma diagnosis and can be utilized as a target for leukemia and lymphoma immunotherapies.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematological malignancy (blood tumor).

The term “pharmaceutical composition” refers to a preparation which is in such form as to permit the biological activity of an active ingredient and/or maintain or improve viability of a biological entity (e.g., a cell) contained therein to be effective in treating a subject, and which contains no additional components, which are unacceptably toxic to the subject in the amounts provided in the pharmaceutical composition.

The term “pharmaceutically acceptable carrier” includes saline, solvents, dispersion media, coatings, antibacterial and antifungal agents, isotonic and absorption delaying agents, and the like, compatible with pharmaceutical administration. In some embodiments, the pharmaceutically acceptable carrier is phosphate buffered saline, saline, Krebs buffer, Tyrode's solution, contrast media, or omnipaque, or a mixture thereof. The term “pharmaceutically acceptable carrier” includes also sterile mitochondria buffer (300 mM sucrose; 10 mM K+-HEPES (potassium buffered (4-(2-hydroxyethyl)-1-piperazineethanesulfonic acid, pH 7.2); 1 mM K+-EGTA, (potassium buffered ethylene glycol tetraacetic acid, pH 8.0)). The term further includes a respiration buffer (250 mM sucrose, 2 mM KH2PO4, 10 mM MgCh, 20 mM K-15 HEPES Buffer (pH 7.2), and 0.5 mM K-EGTA (pH 8.0)). The term further includes a T cell medium, e.g., RPMI 1640 medium GlutaMAX™ Supplement 500 ml (ThermoFisher, 61870010).

The terms “modulate” and “modulation” refer to reducing or inhibiting or, alternatively, activating or increasing, a recited variable.

The terms “increase”, “activate” and “enhance” refer to an increase of 5%, 10%, 15%, 20%, 25%, 30%, 35%, 40%, 45%, 50%, 55%, 60%, 65%, 70%, 75%, 80%, 85%, 87%, 90%, 95%, 98%, 99%, 100%, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 15-fold, 20-fold, 25-fold, 30-fold, 35-fold, 40-fold, 45-fold, 50-fold, 60-fold, 70-fold, 80-fold, 90-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The term “immune cells” refers to cells belonging to the immune system to protect the organism from diseases such as infections or cancers. “Immune cells” are classified between the innate and adaptive immune response.

The term “population of immune cells” or “population of adaptive immune cells” refers to an heterogenous group of immune cells or adaptive immune cells.

The term “effector T cell” and “memory T cell” includes T helper (i.e., CD4*) cells and cytotoxic (i.e., CD8⁺) T cells. CD4⁺ effector T cells typically contribute to the development of several immunologic processes, including maturation of B cells into plasma cells and memory B cells, and activation of cytotoxic T cells and macrophages. CD8⁺ effector T cells typically kill virus-infected cells and tumor cells. CD8⁺ memory T cells typically provide long-term protection against re-infection or cancer relapses through enhanced recall capacity. See Seder and Ahmed, 2003, incorporated by reference in its entirety, for additional information on effector T cells (Seder and Ahmed, 2003, “Similarities and differences in CD4+ and CD8+ effector and memory T cell generation”, Nat Immunol 4:835-42). The CD8 effector memory cells express the surface markers CD62L−, CD45RA+, CD45RO−.

The term “central memory T cells” (“Tcm cells”) refers to cells that express the surface markers CD62L+, CD45RA−, CD45RO+. Human Tcm cells that constitutively express CD62L, which is required for cell extravasation through high endothelial venules (HEV) and migration to T cell areas of secondary lymphoid organs. Tcm cells efficiently mediate recall responses upon encountering a second time the identical antigen.

The term “effector memory cells”, such as CD8 effector memory cells, refers to cells which express the surface markers CD62L−, CD45RA−, CD45RO+, are able to migrate to inflamed peripheral tissues and display effector functions.

The term “memory-like cell”, such as “memory-like T cells”, refers to cells which display characteristics and properties of memory cells. They may mimic one or more surface marker expression, display at least one or more hallmarks of memory cells, such as enhanced recall capacity, survival, self-renewal. For instance, in presence of an acute infection, a T cell can be classified as memory depending on its surface expression and behavior. Some T cells will display memory-like properties during an antitumor response (Shiki Takamura, International Immunology, Volume 32, Issue 9, 1 Sep. 2020, Pages 571-581).

The term “Tscm cell” or “stem cell-like memory cells” refers to a memory cell in its earliest and long-lasting developmental stage, displaying stem cell-like properties, and exhibiting a gene profile between naïve and central memory T cell. Stem cell-like memory cells express the surface markers CD62L+, CD45RA+, CD45RO+.

The term “Trm cell” or “tissue-resident T cell” refers to a subset of a long-lived memory T cells that occupies epithelial and mucosal tissues (skin, mucosa, lung, brain, pancreas, gastrointestinal tract) without recirculating. Trm cells are transcriptionally, phenotypically and functionally distinct from central memory (T_{CM}) and effector memory (T_{EM}) T cells which recirculate between blood, the T cell zones of secondary lymphoid organ, lymph and nonlymphoid tissues. Trm cells themself represent a diverse populations because of the specializations for the resident tissues.

The term “naïve cells” refers to cells, which are resting cells and have not been activated. for example, naïve T cells have not encountered their antigen and circulate in the organism to screen peptides presented by APCs. Naïve T cells express the surface markers CD62L+, CD45RA+, CD45RO−.

The term “adaptive immune cell” refers to cells belonging to the “adaptive immune system” or “acquired immune system” and playing a crucial role in the adaptive immunity. The adaptive immune cells are a subset of the immune cells, They are highly specialized systemic cells. In particular, Lymphocytes B cells and T cells are adaptive immune cells. The adaptive immune cells can have the function of eliminating pathogens or prevent their growth. Adaptive immunity can create immunological memory (e.g. memory B cells or memory T cells) after an initial response to a specific pathogen, and leads to an enhanced response to future encounters with that pathogen. The term “adaptive immune cells” and “adaptive cells” can be used interchangeably.

The term “CD4 T cell” and “CD8 T cell” refer to CD4-positive T cell and CD8-positive T cell respectively. The terms “CD4 T cell”, “CD4 immune T cell”, and “CD4 immune cell” can be used interchangeably. The terms “CD8 T cell”, “CD8 immune T cell” and “CD8 immune cell” can be used interchangeably.

The term “regulatory T cell” or “Treg” includes cells that regulate immunological tolerance, for example, by suppressing effector T cells. In some aspects, the regulatory T cell has a CD4⁺CD25⁺Foxp3⁺ phenotype. In some aspects, the regulatory T cell has a CD8⁺CD25⁺ phenotype. See Nocentini et al., Br. J. Pharmacol., 2012, 165:2089-2099, incorporated by reference in its entirety, for additional information on regulatory T cells. As used herein, the term “regulatory T cells (Tregs)” preferably indicates a subset of CD4+ T cells that are crucial for immune homeostasis. Tregs are defined by their expression of the transcription factor forkhead-box protein P3 (Foxp3), which is essential for their development and suppressive function. Loss of Foxp3 function leads to severe lymphoproliferative disease and autoimmunity. In addition to preventing autoimmunity and inflammatory diseases, Tregs ensure a controlled immune response upon pathogen encounter and thereby prevent immune pathology. Conversely, excessive suppression by Tregs can hamper pathogen clearance and promote chronic infection. In addition, Tregs can also restrain anti-tumor immune responses and thus promote tumor progression.

The term “dendritic cell” refers to a professional antigen-presenting cell capable of activating a naïve T cell and stimulating growth and differentiation of a B cell.

The phrase “disease associated with expression of [target]” includes, but is not limited to, a disease associated with expression of [target] or condition associated with cells which express [target] including, e.g., proliferative diseases such as a cancer or malignancy or a precancerous condition, such as solid tumor or hematological tumor. Non-cancer related indications associated with expression of [target] include, but are not limited to, e.g., autoimmune disease, (e.g., lupus, rheumatoid arthritis, colitis), inflammatory disorders (allergy and asthma), and transplantation.

The term “stimulation” refers to a primary response induced by binding of a stimulatory domain or stimulatory molecule (e.g., a CAR or a TCR/CD3 complex) with its cognate ligand or antigen-independent CD3/CD28 beads when in vitro, thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell or an engineered immune cell (e.g., an immune cell engineered to express a CAR) that provides the primary cytoplasmic signaling sequence(s) that regulate primary activation of a TCR/CAR complex in a stimulatory way for at least some aspect of a signaling pathway, such as a T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. In one aspect, the primary signal is initiated by, for instance, binding of a CAR (e.g., an antibody fragment or chimeric TCR) to its cognate antigen or epitope.

The term “antigen presenting cell” or “APC” refers to an immune system cell such as an accessory cell (e.g., a dendritic cell, a macrophage, and the like) that displays a foreign antigen complexed with major histocompatibility complexes (MHC's) on its surface. T cells may recognize these complexes using their T cell receptors (TCRs). APCs typically process antigens and present them to T cells, but may also be “loaded” with preprocessed antigenic peptides.

An “intracellular signaling domain,” as the term is used herein, refers to an intracellular portion of a molecule involved in generating a signal that promotes an immune effector function, such as the effector function of a TCR- or CAR-expressing T cell. The term “costimulatory molecule” refers to the cognate binding partner on a T cell that specifically binds with a costimulatory ligand, thereby mediating a costimulatory response by the T cell, such as, but not limited to, proliferation.

Costimulatory molecules are cell surface molecules other than antigen receptors or their ligands that may be required for an efficient immune response. Costimulatory molecules include, but are not limited to, an MHC class I molecule, BTLA and a Toll ligand receptor, as well as DAP10, DAP12, CD30, LIGHT, OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g. mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain one or more introns.

The term “endogenous” refers to any material from or produced inside an organism, cell, tissue or system.

The term “exogenous” refers to any material introduced from or produced outside an organism, cell, tissue or system. In case of a patient, the term “exogenous” may refer to patient-, donor- or cell culture-derived material. For example, mitochondria isolated from the patients' muscle tissue and subsequently introduced to a population of immune cells, which may be autologous to the patient or autogenic, are considered exogenous. The term “exogenous mitochondria” refers to any mitochondria isolated from an autogenous source, an allogeneic source, and/or a xenogeneic source, wherein the source's nature may be of tissue, blood, or cultured cells.

The term “expression” refers to the transcription and/or translation of a particular nucleotide sequence driven by a promoter.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “expression construct” or “transgene” is defined as any type of genetic construct containing a nucleic acid coding for gene products in which part or all of the nucleic acid encoding sequence is capable of being transcribed can be inserted into the vector.

As used herein with reference to a disease, disorder or condition, the terms “treatment”, “treat”, “treated”, or “treating” refer to prophylaxis and/or therapy.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses

The term “lentiviral vector” refers to a vector derived from at least a portion of a lentivirus genome, including especially a self-inactivating lentiviral vector as provided in Milone et al., Mol. Ther. 17(8): 1453-1464 (2009).

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. The term “parenteral” administration of an immunogenic composition includes, e.g., subcutaneous (s.c.), intravenous (i.v.), intramuscular (i.m.), intranasal or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR) and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

The term “promoter” refers to a DNA sequence recognized by the transcription machinery of the cell, or introduced synthetic machinery, that can initiate the specific transcription of a polynucleotide sequence.

The term “promoter/regulatory sequence” refers to a nucleic acid sequence which can be used for expression of a gene product operably linked to the promoter/regulatory sequence. In some instances, this sequence may be the core promoter sequence and in other instances, this sequence may include an enhancer sequence and other regulatory elements, which are required for expression of the gene product. The promoter/regulatory sequence may, for example, be one, which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term, a “substantially purified” cell refers to a cell that is essentially free of other cell types. A substantially purified cell also refers to a cell, which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” refers to antigens that are common to specific hyperproliferative disorders. In certain aspects, the hyperproliferative disorder antigens of the present invention are derived from, cancers including but not limited to primary or metastatic melanoma, mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, and stomach cancer.

The term “transfected” or “transformed” or “transduced” refers to a process by which exogenous nucleic acid is transferred or introduced into the host cell. A “transfected” or “transformed” or “transduced” cell is one, which has been transfected, transformed or transduced with exogenous nucleic acid. The cell includes the primary subject cell and its progeny.

The term “T cell exhaustion” and “exhausted T cell” refers to either hyporesponsive T cells or “dysfunctional” T cells.

The term “activity” or “activity of an immune cell” refers to cell effector function, such as cytotoxic activity towards the target cell expressing a certain antigen and detected by the TCR specific for that antigen or cytokine production. It further refers to metabolic activity, proliferative capacity and ability to expand and divide, capacity to resist to exhaustion, and suppressive activity.

The term “survival” of a cell or a population of cells refers to, but it is not limited to, cell persistence, cell self-renewal capacity, cell endurance. Cell survival can be defined as the process that encompasses the viability of a cell and its ability to subsist and maintain the integrity of cellular processes. Survival mechanisms ensure that the cell will be able to adapt and carry-on cellular activities such as replication, repair, and metabolism.

The term “enhancement of the survival” of immune cells, such as T cells, indicates, for instance, the enhancement of one or more of the following properties of the immune cells: (i) the ability to survive in resting phase and upon re-stimulation; (ii) the responsiveness to interleukins: such as IL-7, IL-15 (e.g., the cells may need less signals to survive as they should increase their expression of receptor to respond to those interleukins); (iii) the resistance to activation-induced cell death (AICD); (iv) the resistance to apoptosis by upregulating anti-apoptotic molecules, such as BCL-XL, BCL-2, etc.; (v) the resistance to apoptosis by downregulating pro-apoptotic molecules, such as Fas and FasL expression; (vi) the epigenetic modifications and transcriptional alterations, which may influence the expression of certain genes, such as Bcl2, Bcl212, Mcl1, Bcl2ald, Birc2, Birc3, Xiap, Cflar; (viii) lengths of telomeres.

The term “promoting the selection” refers to the increase in amount and/or the enhancement of some properties of one or more subsets of immune cells over the bulk of immune cells or immune cell population.

The term “differentiation” or “cell/cellular differentiation” refers to the process of a cell changing from one cell type to another, typically, but not only, from a less specialized type (stem cell) to a more specialized type. Differentiation, especially the immune cell differentiation, can occur in response to antigen exposure. Differentiation may dramatically change a cell's size, shape, membrane potential, metabolic activity, and responsiveness to signals.

The term “immune cells treated with mitochondria” may refer to immune cells exposed to, having been in close contact with, co-incubated with or transplanted with mitochondria. The term “mitochondrial treatment” refers to the act to expose the cells to mitochondria, or to the act of putting/placing the cells in close contact with the mitochondria, or to the act of co-incubating the cells with mitochondria, or to the act of transplanting the mitochondria into cells.

The term “self-renewal capacity” or “cell self-renewal capacity” refers to the cell process of giving rise to indefinitely more cells of the same cell type. Self-renewal is the capacity to divide and retain all the features of the mother cell. Self-renewal leaves the number of cells roughly the same.

The term “recall capacity” refers to a secondary immune response mounted by memory cells leading to a quick proliferation and differentiation into effector cells. This rapid recall response is critical in controlling the extent of infection and preventing disease.

The term “transplanted mitochondria” refers to exogenous mitochondria substantially integrated in the target cell (e.g. partially or fully integrated into the cell). The term “mitochondrial transplantation”, “transplantation of mitochondria” or “transfer of mitochondria” refers to the act of integrating/transferring exogenous mitochondria into a host cell.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Enhanced Immune Cells and Compositions Related Thereto

In the following the invention is described in more detail. In particular, the invention relates to the following items:
1. The present disclosure provides immune cells, e.g. human immune cells, treated with isolated viable mitochondria in an amount effective to enhance the survival and/or to promote the selection of adaptive immune cells, e.g. human adaptive immune cells, such as B cells or T cells, preferably T cells, such as CD8 immune cells or CD4 immune cells, relative to adaptive immune cells, e.g. human adaptive immune cells, not treated with isolated viable mitochondria.
2. The present disclosure further provides immune cells, e.g. human immune cells, treated with isolated viable mitochondria in an amount effective to promote memory cell differentiation and/or memory cell selection of adaptive immune cells, e.g. memory T cells, relative to immune cells not treated with isolated viable mitochondria.
3. In particular, the present disclosure provides immune cells, e.g. human immune cells, such as human immune T cells, treated with isolated viable mitochondria in an amount effective to enhance the survival and/or to promote the selection of memory CD8 T cells relative to immune cells, e.g. human adaptive immune cells, not treated with isolated viable mitochondria. In some particular aspects, the isolated viable mitochondria are in an amount effective to enhance the survival and/or to promote the selection of central memory CD8 T cells relative to immune cells, e.g. human adaptive immune cells, not treated with isolated viable mitochondria. In some other particular aspects, the isolated viable mitochondria are in an amount effective to enhance the survival and/or to promote the selection of effector memory CD8 T cells relative to immune cells not treated with isolated viable mitochondria.
4. In particular, the present disclosure provides immune cells, e.g. human immune cells, such as human T immune cells, treated with isolated viable mitochondria in an amount effective to enhance the survival and/or to promote the selection of Treg cells relative to immune cells, e.g. human adaptive immune cells, such as CD4 immune cells, not treated with isolated viable mitochondria.
5. The present disclosure also provides immune cells, e.g. human immune cells, comprising exogenous isolated viable mitochondria (e.g. comprising mitochondria which are partially of fully integrated into the cell), in an amount effective to enhance the survival and/or to promote the selection of adaptive immune cells, e.g. human adaptive immune cells, such as B cells or T cells, preferably T cells, such as CD8 or CD4 immune cells, relative to adaptive immune cells, e.g. human adaptive immune cells, not comprising exogenous isolated viable mitochondria.
6. The present disclosure further provides immune cells, e.g. human immune cells, such as human immune T cells, comprising exogenous isolated viable mitochondria in an amount effective to promote memory cell differentiation and/or memory cell selection of adaptive immune cells, e.g. memory T cells, relative to immune cells, e.g. human adaptive immune cells, not comprising exogenous isolated viable mitochondria.
7. In particular, the present disclosure provides immune cells, e.g. human immune cells, such as human T immune cells, comprising exogenous isolated viable mitochondria in an amount effective to enhance the survival and/or to promote the selection of memory CD8 T cells relative to immune cells e.g. human adaptive immune cells, not comprising isolated viable mitochondria. In some particular aspects, the isolated viable mitochondria are in an amount effective to enhance the survival and/or to promote the selection of central memory CD8 T cells relative to immune cells not comprising exogenous isolated viable mitochondria. In some other particular aspects, the isolated viable mitochondria are in an amount effective to enhance the survival and/or to promote the selection of effector memory CD8 T cells relative to immune cells not comprising isolated viable mitochondria.
8. In particular, the present disclosure provides immune cells, e.g. human immune cells, such as human T immune cells, comprising exogenous isolated viable mitochondria in an amount effective to enhance the survival and/or to promote the selection of Treg cells relative to immune cells, such as human adaptive immune cells, e.g. CD4 immune cells, not comprising exogenous isolated viable mitochondria.
9. Also provided for herein is a population comprising the immune cells, such as human immune cells, e.g. human adaptive immune cells, according to any one of the preceding items.
10. The present disclosure also provides a composition comprising immune cells or a composition of immune cells, such as human immune cells, e.g. human adaptive immune cells, treated with isolated viable mitochondria in an amount effective to enhance the survival and/or to promote the selection of adaptive immune cells, e.g. human adaptive immune cells, such as B cells or T cells, preferably T cells, such as CD8 immune cells or CD4 immune cells, relative to adaptive immune cells, e.g. human adaptive immune cells, not treated with isolated viable mitochondria. In particular, the composition comprises immune cells, e.g. human immune cells, treated with isolated viable mitochondria in an amount effective to enhance the survival and/or to promote the selection of memory CD8 T cells, such as central memory CD8 T, effector memory CD8 T cells, or combination thereof, relative to immune cells, e.g. human adaptive immune cells, not treated with isolated viable mitochondria. In particular, the compositions comprises immune cells, such as human immune cells, e.g. human T cells, treated with isolated viable mitochondria in an amount effective to enhance the survival and/or to promote the selection of Treg cells relative to immune cells, e.g. human adaptive immune cells, such as CD4 immune cells, not treated with isolated viable mitochondria.
11. The present disclosure provides a composition comprising immune cells or a composition of immune cells, e.g. human immune cells, treated with isolated viable mitochondria in an amount effective to promote memory cell differentiation and/or memory cell selection of adaptive immune cells, e.g. memory T cells, relative to immune cells not treated with isolated viable mitochondria.
12. The present disclosure provides a composition comprising immune cells or a composition of immune cells, e.g. human immune cells, wherein the immune cells comprise exogenous isolated viable mitochondria in an amount effective to enhance the survival and/or to promote the selection of adaptive immune cells, e.g. human adaptive immune cells, such as B cells or T cells, preferably T cells, such as CD8 immune cells or CD4 immune cells, relative to adaptive immune cells, e.g. human adaptive immune cells, not comprising exogenous isolated viable mitochondria. In particular, the composition comprises immune cells, e.g. human immune cells, comprising exogenous isolated viable mitochondria in an amount effective to enhance the survival and/or to promote the selection of memory CD8 T cells, such as central memory CD8 T cells, effector memory CD8 T cells, or combination thereof, relative to immune cells, e.g. human adaptive immune cells, not comprising exogenous isolated viable mitochondria. In particular, the composition comprises immune cells, e.g. human immune cells, comprising exogenous isolated viable mitochondria in an amount effective to enhance the survival and/or to promote the selection of Treg cells relative to immune cells, e.g. human immune cells, such as CD4 immune cells, not comprising exogenous isolated viable mitochondria.
13. The present disclosure provides a composition comprising immune cells or a composition of immune cells, e.g. human immune cells, wherein the immune cells comprise exogenous isolated viable mitochondria in an amount effective to promote memory cell differentiation and/or memory cell selection of adaptive immune cells, e.g. memory T cells, relative to immune cells not comprising isolated viable mitochondria.
14. The present disclosure provides a composition comprising immune cells or a composition of immune cells, e.g. human immune cells, treated with isolated viable mitochondria in an amount effective to enhance the survival and/or promote the selection of a population of adaptive immune cells, e.g. a population of human adaptive immune cells, such as B cells or T cells, preferably T cells, such as CD8 immune cells or CD4 immune cells, relative to a population of immune cells, e.g. human immune cells, not treated with mitochondria.
15. The present disclosure provides a composition comprising immune cells or a composition of immune cells, e.g. human immune cells, treated with isolated viable mitochondria in an amount effective to enhance memory cell differentiation and/or promote memory cell selection of a population of adaptive immune cells, e.g. memory T cells, relative to a population of immune cells not treated with isolated viable mitochondria.
16. In particular, the present disclosure provides a composition comprising immune cells, e.g. human immune cells, such as human T cells, treated with isolated viable mitochondria in an amount effective to enhance the survival and/or to promote the selection of a population of memory CD8 T cells, such as central memory CD8 T cells, effector memory CD8 T cells, or a combination thereof, relative to immune cells, e.g. adaptive immune cells, not treated with isolated viable mitochondria. In some aspects, the mitochondria are capable of enhancing the proportion of the memory adaptive cells, e.g. central memory CD8 T cells or effector memory CD8 T cells, of at least 20%, preferably of a last 30%, more preferably of at least 50%.
17. The present disclosure also provides a composition comprising immune cells or a composition of immune cells, e.g. human immune cells, wherein the immune cells comprise exogenous isolated viable mitochondria in an amount effective to enhance the survival and/or promote the selection of a population of adaptive immune cells, e.g. a population of human adaptive immune cells, such as B cells or T cells, preferably T cells, such as CD8 immune cells or CD4 immune cells, relative to a population of immune cells, e.g. human immune cells, not comprising exogenous isolated mitochondria.
18. The present disclosure provides a composition comprising immune cells or a composition of immune cells, e.g. human immune cells, wherein the immune cells comprise exogenous isolated viable mitochondria in an amount effective to enhance memory cell differentiation and/or promote memory cell selection of adaptive immune cells within a population of adaptive immune cells relative to a population of immune cells, e.g. memory T cells, not comprising exogenous mitochondria.
19. In particular, the present disclosure provides a composition comprising immune cells, e.g. human immune cells, such as human T cells, which comprise exogenous isolated viable mitochondria in an amount effective to enhance the survival and/or to promote the selection of a population of memory CD8 T cells, such as central memory CD8 T cells, effector memory CD8 T cells, or a combination thereof, relative to immune cells, e.g. adaptive immune cells, not comprising exogenous isolated viable mitochondria. In some aspects, the mitochondria are capable of enhancing the proportion of the memory adaptive cells, e.g. central memory CD8 T cells or effector memory CD8T cells, of at least 20%, preferably of at least 30%, more preferably of at least 50%.
20. The compositions according to any one of the preceding items can be formulated in solid or liquid form, preferably in liquid form.
21. In some aspects, the survival of the adaptive immune cells, such as memory immune cells (e.g. central memory CD8 T cell and effector memory CD8 T cells) or memory-like cells, according to any one of the preceding items is the self-renewal capacity of the adaptive immune cell. In some other aspects, the enhanced survival of the adaptive immune cells, whose selection has been promoted by the mitochondria according to any one of the preceding items, is an improved self-renewal capacity.
22. In some aspects, the survival of the adaptive immune cells, such as memory immune cells (e.g. central memory CD8 T cell and effector memory CD8 T cells) or memory-like immune cells, according to any one of the preceding items is the survival capacity of the adaptive immune cells in resting phase and upon re-stimulation. In some other aspects, the enhanced survival of the adaptive immune cells, whose selection has been promoted by the mitochondria according to any one of the preceding items, is an improved survival capacity in resting phase and upon re-stimulation.
23. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to respond to interleukin signaling, such as the signaling of IL-7 and/or IL-15. In some other aspects, the enhanced survival of the adaptive immune cells, whose selection has been promoted by the mitochondria according to any one of the preceding items, is an improved capacity to respond to interleukin signaling, such as the signaling of IL-7 and/or IL-15.
24. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to resist to activation-induced cell death (AICD). In some other aspects, the enhanced survival of the adaptive immune cells consists in the improved capacity of the adaptive immune cells, whose selection has been promoted by the mitochondria according to any one of the preceding items, is an improved capacity to resist to activation-induced cell death (AICD) of the adaptive immune.
25. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to resist to apoptosis by upregulating anti-apoptotic molecules, such as BCL-XL, BCL-2, or by downregulating pro-apoptotic molecules, such as Fas and FasL expression. In some aspects, the enhanced survival of the adaptive immune cells, whose selection has been promoted by the mitochondria according to any one of the preceding items, consists in an improved capacity to resist to apoptosis by upregulating anti-apoptotic molecules, such as BCL-XL, BCL-2, or by downregulating pro-apoptotic molecules, such as Fas and FasL expression.
26. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to express epigenetic modifications and transcriptional alterations, which influence the expression of genes selected from the group consisting of Bcl2, Bcl212, Mcl1, Bcl2ald, Birc2, Birc3, Xiap and Cflar. In some aspects, the enhanced survival of the adaptive immune cells, whose selection has been promoted by the mitochondria according to any one of the preceding items, consists in an improved capacity of producing epigenetic modifications and transcriptional alterations, which influence the expression of genes selected from the group consisting of Bcl2, Bcl212, Mcl1, Bcl2ald, Birc2, Birc3, Xiap and Cflar.
27. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to maintain their long telomeres. In some aspects, the enhanced survival of the adaptive immune cells, whose selection has been promoted by the mitochondria according to any one of the preceding items, consists in an improved capacity of maintaining long telomeres relative to immune cells not treated with isolated viable mitochondria or not comprising exogenous isolated viable mitochondria.
28. In some aspects, the survival of the adaptive immune cells, such as memory immune cells (e.g. central memory CD8 T cell and effector memory CD8 T cells) or memory-like immune cells, according to any one of the preceding items consists in the capacity of the adaptive immune cells to proliferate or to exhibit persistence, or a combination thereof, relative to immune cells not treated with isolated viable mitochondria or not comprising exogenous mitochondria.
29. In some aspects, the survival of the adaptive immune cells, e.g. memory immune cells (e.g. central memory CD8 T cell and effector memory CD8 T cells) or memory-like immune cells, according to any one of the preceding items consists in a diminished exhaustion of the adaptive immune cells, relative to immune cells not treated with isolate viable mitochondria or not comprising exogenous mitochondria.
30. In some aspects, the compositions of any one of the preceding items are pharmaceutical compositions. In some other aspects, the pharmaceutical compositions further comprise at least one pharmaceutically acceptable carrier. In some aspects, the pharmaceutically acceptable carrier is formulated for delivery into a human immune cell. In some aspects, the pharmaceutically acceptable carrier is formulated for delivery into human tissues and/or organs. The pharmaceutically acceptable carrier includes, but is not limited to, saline, dispersion media, isotonic agents, and the like, compatible with pharmaceutical administration. In some aspects, the pharmaceutically acceptable carrier is phosphate buffered saline, saline, Krebs buffer, Tyrode's solution, contrast media, or omnipaque, or a mixture thereof. In some other aspects, the carrier is a buffer comprising 300 mM sucrose; 10 mM K+-HEPES (potassium buffered (4-(2-hydroxyethyl)-1-piperazineethanesulfonic acid, pH 7.2); 1 mM K+-EGTA, (potassium buffered ethylene glycol tetraacetic acid, pH 8.0), a buffer comprising 250 mM sucrose, 2 mM KH2PO4, 10 mM MgCh, 20 mM K-15 HEPES Buffer (pH 7.2), and 0.5 mM K-EGTA (pH 8.0), or RPMI 1640 medium GlutaMAX™ Supplement 500 ml (ThermoFisher, 61870010).
31. In some aspects, the immune cells, e.g. human immune cells, such as adaptive immune cells, according to any of the preceding items are derived from a biological sample selected from the group: blood and other liquid samples of biological origin, solid tissue samples, tissue culture of cells derived therefrom and the progeny thereof, isolated cells from biological samples.
32. In some aspects, the immune cells of any one of the preceding items are produced from viable eukaryotic cells. In some other aspects, the immune cells are produced in vitro or ex vivo.
33. In some aspects, the immune cells, e.g. human immune cells, such as adaptive immune cells, according to any of the preceding items allogeneic or autologous immune cells. In some aspects the immune cells are xenogeneic.
34. In some aspects, the immune cells, e.g. human immune cells, such as adaptive immune cells, according to any of the preceding items are produced from a stem cell comprising or a mesenchymal stem cell or an induced pluripotent stem cell (iPSC).
35. In some aspects, the immune cells according to any of the preceding items are preferably mammalian immune cells, more preferably human immune cells.
36. In some aspects the immune cells, may include, but are not limited to, engineered or propagated in vitro natural immune cells.
37. In some aspects, the immune cells according to any one of the preceding items are preferably adaptive immune cells, such as B lymphocytes or T lymphocytes, preferably T lymphocytes. In some aspects, the adaptive immune cells are propagated in vitro B lymphocytes or T lymphocytes, preferably propagated in vitro T lymphocytes.
38. In some aspects, the immune cells according to any one of the preceding embodiments are T Lymphocytes, such as not limited to, alpha-beta T cells (αβT cells), gamma-delta T cells (γδT cells), CD4 immune cells, CD8 immune cells, or a combination thereof. In some aspects, the T lymphocytes are naïve T cells, effector T cells, memory T cells (e.g., tissue resident memory (Trm) cells, Tscm cells, central memory cells and effector memory cells), memory-like T cells, or a combination thereof. In some other aspects, the T cells are preferably memory T cells. In some aspects, the immune cells, e.g. human immune cells, according to any one of the preceding items are a pluripotent stem cell-derived immune cell. In some other aspects, the T lymphocytes are helper T cells (TH), cytotoxic T cells (CTLs), regulatory T (Treg) cells, memory T cells, or a combination thereof. In some other aspects, the T cells are preferably regulatory T cells (Treg). In some aspects, the immune cells are mucosal associated invariant T cells. In some other aspects, the immune cells are T cells circulating in the blood or tumor-infiltrating lymphocytes (TILs).
39. In some aspects, the immune cells according to any of the preceding items are preferably CD8 T cells, such as but not limited to, CD8 T cells circulating in the blood, tumor-infiltrating lymphocytes (TILs), e.g. CD8 TILs, naïve CD8 T cells, effector CD8 T cells, memory CD8 T cells, (e.g. Trm CD8 T cells, Tscm CD8 T cells, central memory CD8 T cells and effector memory CD8 T cells), memory-like CD8 T cells, or combination thereof. In some preferred aspects, the CD8 T cells are TILs. In some more preferred aspects, the immune cells are memory CD8 T cells, in particular effector memory CD8 T cells, central memory CD8 T cells, or a combination thereof. In some preferred aspects, the immune cells are memory-like CD8 T cells.
40. In some aspects, the T cells are CD4 T cells, such as naïve CD4 T cells, effector CD4 T cells (e.g., Th1, Th2, or Th17), memory CD4 T cells, memory-like CD4 T cells, regulatory CD4 T cells (Treg), CD4 T cells circulating in the blood, tumor infiltrating lymphocytes (TIL) CD4 T cells, or a combination thereof. Preferably the CD4 T cells are Treg cells, e.g. human regulatory (Treg) CD4 T cells.
41. In some aspects, the immune cells, e.g. human immune cells, of any of the preceding items, may include, but are not limited to, engineered immune cells expressing a chimeric antigen receptor (“CAR”) and/or artificial T-cell receptor (“TCR”) subunit, such as but not limited to CAR-T cells, e.g. CD8 CAR-T cells. CAR T cells typically include an antigen-binding moiety (e.g., an antigen-binding domain or antigen-binding fragment thereof), a transmembrane component, and a primary cytoplasmic signaling sequence selected to activate the immune cell in response to the antigen-binding moiety binding its cognate ligand. In some aspects, the basic components of a chimeric antigen receptor (CAR) include the following: (1) The variable heavy (V_{H}) and light (V_{L}) chains for a tumor-specific monoclonal antibody are fused in-frame with the CD3ζ-chain from the T cell receptor complex. (2) The V_{H} and V_{L} are generally connected together using a flexible glycine-serine linker, and then attached to the transmembrane domain by a spacer (e.g., CD8a stalk or C_{H}2-C_{H}3 constant domains) to extend the scFv away from the cell surface so that it can readily interact with tumor antigens. In some embodiments, the engineered immune cell comprising exogenous mitochondria is CAR-T cell.
42. In some aspects, the CAR or artificial TCR subunit is introduced into the immune cells using a virus, such as a lentivirus or adenovirus or retrovirus, nanoparticle, or a nanoparticle operably connected to a targeting moiety. In some aspects, the exogenous polynucleotide encoding the CAR and/or artificial TCR subunit is introduced into the immune cells in vitro. In some aspects, the vector is a viral vector. In some aspects, the viral vector is derived from a retrovirus, lentivirus, adenovirus, adeno-associated virus, or hybrid vector.
43. In some aspects, the immune cells or population of immune cells according to any one of the preceding items, comprise a CAR or artificial TCR subunit comprising an antigen selected from the group: B-cell maturation antigen (BCMA, also known as tumor necrosis factor receptor superfamily member 17, TNFRSF17), CD19, CD123, CD22, CD30, CD171, CS-1 (also referred to as CD2 subset 1, CRACC, SLAMF7, CD319, and 19A24), C-type lectin-like molecule-1 (CLL-1 or CLECL1), CD33, epidermal growth factor receptor variant III (EGFRvIII), ganglioside G2 (GD2), ganglioside GD3, Tn antigen (Tn Ag or GalNAca-Ser/Thr), prostate-specific membrane antigen (PSMA), receptor tyrosine kinase-like orphan receptor 1 (ROR1), Fms-Like Tyrosine Kinase 3 (FLT3), tumor-associated glycoprotein 72 (TAG72), CD38, CD44v6, carcinoembryonic antigen (CEA), epithelial cell adhesion molecule (EpCAM), B7H3 (CD276), KIT (CD117), interleukin-13 receptor subunit alpha-2 (IL-13Ra2 or CD213A2); mesothelin, interleukin 11 receptor alpha (IL-11Ra), prostate stem cell antigen (PSCA), protease Serine 21 (Testisin or PRSS21), vascular endothelial growth factor receptor 2 (VEGFR2), Lewis Y antigen, CD24, platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl)(bcr-abl); tyrosinase; ephrin type-A receptor 2 (EphA2); Fucosyl GM1; sialyl Lewis adhesion molecule (sLe); ganglioside GM3 (aNeu5Ac(2-3)bDG alp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some aspects, the immune cells or population of immune cells of any one of the preceding items comprises a CAR of first, second, third or fourth generation.
44. In some aspects, the specific lymphocyte activating receptor agonist of any one of the immune cells of the preceding items is conjugated to cell mimicking cell-free supports. In some aspects, the cell-mimicking supports are paramagnetic beads.
45. In some aspects, the immune cells of any one of the preceding items are cells, e.g. effector cells, known in the art with anti-tumor or immunosuppressive and activity. In some other aspects, the immune cells are cells with an immunoregulating activity.
46. The isolated viable mitochondria of any one of the preceding items are preferably respiration competent mitochondria.
47. The effective amount of the isolated viable mitochondria according to any one of the preceding items is between 0.0001 ng and 2.5 ng of mitochondria per target cell, e.g. between 0.001 ng and 2.0 ng, such as, for example, between 0.01 ng and 1.5 ng or between 0.05 ng and 1.0 ng, e.g. between 0.1 ng and 0.5 ng of mitochondria per target cell.
48. The isolated viable mitochondria of any one of the preceding items can be autologous, or allogeneic mitochondria. In some other aspects, they are xenogeneic mitochondria.
49. In some other aspects, the isolated viable mitochondria of any one of the preceding items may be freshly isolated or previously isolated and subsequently stored until use, e.g. stored at a temperature below 0° C.
50. In some aspects, the sources of the isolated viable mitochondria of any one of the preceding items may be of different nature—e.g., tissue, blood, more specifically cells circulating in the blood, or cultured cells.
51. In some aspects, the isolated viable mitochondria are eukaryotic cell mitochondria. In some aspects, the mitochondria are derived from a human cell line.
52. In some aspects, the isolated viable mitochondria of any one of the preceding items are derived from a healthy donor. In some aspects, the isolated viable mitochondria are derived from a patient. In some aspects, the patient is a cancer patient. In some other aspects, the patient is a patient suffering from an autoimmune disease. In some other aspects, the patient is a transplanted patient. In some other aspects, the patient is patient suffering from infectious and/or inflammatory diseases.
53. In some aspects, the isolated viable mitochondria may be autogenous or autologous viable mitochondria with genetic modification. In some aspects, the isolated viable mitochondria may be allogeneic viable mitochondria with genetic modification.
54. In some aspects, the isolated viable mitochondria disclosed in any one of the preceding items may be delivered into target cells, e.g. immune cells, both in vitro and in vivo.
55. In some aspects, the isolated viable mitochondria disclosed in any one of the preceding items may be delivered into target organs and/or tissues, such as into the cells of target organs and/or tissues, in vivo or ex vivo.
56. In some aspects, the viable mitochondria are isolated by using one of the isolation methods described hereinafter, each method comprising the step(s) of: (i) isolating the mitochondria from cultured cells, tissues or organs by using an endopeptidase, such as Subtilisin A; or (ii) filtrating the mitochondria through one or more filters; or (i) isolating the mitochondria from cultured cells, tissues or organs by using an endopeptidase, such as Subtilisin A and subsequently (ii) filtrating the mitochondria through one or more filters.
57. In some aspects, the exogenous viable mitochondria are, but not limited to, autologous, or allogeneic mitochondria, genetically engineered mitochondria, or mitochondria encapsulated by a liposome or coupled to specific agents.
58. In some aspects, the mitochondria, e.g. isolated viable mitochondria, according to any one of the preceding items are capable of enhancing the survival and/or promoting the selection of the adaptive immune cells or population of adaptive immune cells, relative, respectively, to immune cells or population of immune cells not treated with mitochondria, starting from day 3 post mitochondrial treatment, such as from day 3.5 or day 4 post mitochondrial treatment, preferably from day 5 post mitochondrial treatment, such as from day 6, day 7 or day 8, more preferably from day 9 post mitochondrial treatment.
59. In some aspects, the mitochondria, e.g. isolated viable mitochondria, of any one of the preceding items are capable of enhancing the survival and/or promoting the selection of the adaptive immune cells or population of adaptive immune cells relative, respectively, to immune cells or population of immune cells not comprising exogenous mitochondria, starting from day 3 post mitochondrial transplantation of mitochondria into the immune cells, such as from day 3.5 or day 4 post mitochondrial transplantation, preferably from day 5 post mitochondrial transplantation, such as from day 6, day 7 or day 8 post mitochondrial transplantation, more preferably from day 9 post mitochondrial transplantation.
60. In some aspects, the enhancement of the survival of the immune cells or population of immune cells, such as the enhancement of the survival of the adaptive immune cells or population of adaptive immune cells selected upon treatment with the isolated viable mitochondria according to any one of the preceding items, is of at least of 1.2-fold relative to immune cells not treated with mitochondria. In some aspects, it is of at least of 1.3-fold, such as at least 1.5-fold or 2-fold relative to immune cells not treated with mitochondria. In some aspects, it is in the range (expressed in folds) of between 1.2-fold to 50-fold, such as 1.2 to 45, 1.2 to 40, 1.2 to 30, 1.2 to 20, 1.2 to 15, 1.2 to 10, 1.2 to 5, 1.2 to 2.5, 1.3 to 50, 1.3 to 40, 1.3 to 30, 1.3 to 20, 1.3 to 10, 1.3 to 5, 1.3 to 3.5, 1.5 to 30, 1.5 to 25, 1.5 to 20, 1.5 to 15, 1.5 to 10, 1.5 to 5, 1.5 to 3, 1.5 to 2.5, 2 to 50, 2 to 40, 2 to 30, 2 to 20, 2 to 15, 2 to 10, 2 to 5, 2 to 4, 3 to 30, 3 to 20, 3 to 10, 3 to 5, 5 to 50, 5 to 40, 5 to 30, 5 to 20, 5 to 10, 5 to 8, 10 to 50, 10 to 40, 10 to 30, 10 to 20, to 15, 15 to 50, 15 to 40, 15 to 30, 15 to 20, 20 to 50, 20 to 30, 20 to 25, 30 to 35, 30 to 40, 30 to 45, 40 to 50, relative, respectively, to immune cells, e.g. adaptive immune cells, or population of immune cells not treated with mitochondria.
61. In some aspects, the enhancement of the survival of the immune cells or population of immune cells, such as the enhancement of the survival of the adaptive immune cells or population of adaptive immune cells selected upon transplantation with exogenous mitochondria according to any one of the preceding items, is of at least of 1.2-fold relative to immune cells not transplanted with exogenous mitochondria. In some aspects, it is of at least of 1.3-fold, such as at least 1.5-fold or 2-fold relative to immune cells not transplanted with exogenous mitochondria. In some aspects, it is in the range (expressed in folds) of between 1.2-fold to 50-fold, such as 1.2 to 45, 1.2 to 40, 1.2 to 30, 1.2 to 20, 1.2 to 15, 1.2 to 10, 1.2 to 5, 1.2 to 2.5, 1.3 to 50, 1.3 to 40, 1.3 to 30, 1.3 to 20, 1.3 to 10, 1.3 to 5, 1.3 to 3.5, 1.5 to 30, 1.5 to 25, 1.5 to 20, 1.5 to 15, 1.5 to 10, 1.5 to 5, 1.5 to 3, 1.5 to 2.5, 2 to 50, 2 to 40, 2 to 30, 2 to 20, 2 to 15, 2 to 10, 2 to 5, 2 to 4, 3 to 30, 3 to 20, 3 to 10, 3 to 5, 5 to 50, 5 to 40, 5 to 30, 5 to 20, 5 to 10, 5 to 8, 10 to 50, 10 to 40, 10 to 30, 10 to 20, to 15, 15 to 50, 15 to 40, 15 to 30, 15 to 20, 20 to 50, 20 to 30, 20 to 25, 30 to 35, 30 to 40, 30 to 45, 40 to 50, relative to immune cells, e.g. adaptive immune cells not comprising (e.g. not transplanted with) exogenous mitochondria.
62. Also provided for herein is a method of enhancing the survival and/or promoting the selection of immune cells or a population of immune cells according to any one of the preceding items, comprising the step of: (a) activating the immune cells in vitro in a cell-free medium with specific activating receptor agonist antibodies capable of driving the adaptive cells (such as T cells) activation; (b) exposing the immune cells to a pharmaceutical composition comprising isolated viable mitochondria for at least 3 days, such as for at least 5 days. In some aspects, the method of enhancing the survival and/or promoting the selection of immune cells or a population of immune cells according to any one of the preceding items comprises alternatively the step (a) activating the immune cells in vitro in a cell-free medium with coated CD3/CD28 beads, optionally in presence of recombinant interleukins, such IL-2; (b) exposing the immune cells to a pharmaceutical composition comprising isolated viable mitochondria for at least 3 days, such as for at least 5 days.
63. Also provided for herein is a method of promoting memory differentiation and/or memory selection of immune cells or a population of immune cells according to any one of the preceding items, comprising the step of: (a) activating the immune cells in vitro in a cell-free medium with specific activating receptor agonist antibodies capable of driving the adaptive cells (such as T cells) activation; (b) exposing the immune cells to a pharmaceutical composition comprising isolated viable mitochondria according to any one of the preceding items, for at least 3 days, such as, example, for at least 5 days. In some aspects, the method of promoting memory differentiation and/or memory selection of immune cells or a population of immune cells according to any one of the preceding items comprises alternatively the step (a) activating the immune cells in vitro in a cell-free medium with coated CD3/CD28 beads, optionally in presence of recombinant interleukins, such IL-2; (b) exposing the immune cells to a pharmaceutical composition comprising isolated viable mitochondria for at least 3 days, such as, for example, for at least 5 days.
64. The pharmaceutical composition used in the methods of any one of the preceding items comprises isolated viable mitochondria, wherein the mitochondria are as disclosed in any one of the preceding items. The effective amount of isolated viable mitochondria comprised in the pharmaceutical composition used in the methods is between 0.0001 ng and 2.5 ng of mitochondria per target cell, e.g. between 0.001 ng and 2.0 ng, such as, for example, between 0.01 ng and 1.5 ng or between 0.05 ng and 1.0 ng, e.g. between 0.1 ng and 0.5 ng of mitochondria per target cell. In some aspects, the pharmaceutical composition used in the methods of any one of the preceding items further comprises one or more pharmaceutically acceptable carrier. The carrier includes, but is not limited to, saline, dispersion media, isotonic agents, and the like, phosphate buffered saline, Krebs buffer, Tyrode's solution, contrast media, omnipaque, a buffer comprising 300 mM sucrose; 10 mM K+-HEPES (potassium buffered (4-(2-hydroxyethyl)-1-piperazineethanesulfonic acid, pH 7.2); 1 mM K+-EGTA, (potassium buffered ethylene glycol tetraacetic acid, pH 8.0), a buffer comprising 250 mM sucrose, 2 mM KH2PO4, 10 mM MgCh, 20 mM K-15 HEPES Buffer (pH 7.2), and 0.5 mM K-EGTA (pH 8.0), or RPMI 1640 medium GlutaMAX™ Supplement 500 ml (ThermoFisher, 61870010). The pharmaceutical composition comprises one or more pharmaceutically acceptable carrier and isolated viable mitochondria in an amount effective to enhance the proportion of adaptive memory or memory-like immune cells relative to immune cells not treated with or not transplanted with mitochondria, of at least 1.1-fold, such of 1.2-fold, 1.3-fold, 1.5-fold, or 2-fold. In some embodiments, the enhancement of the proportion of the memory or memory-like immune cells is in the range (expressed in folds) of between 1.1-fold to 100-fold, such as 1.1 to 99, 1.1 to 90, 1.1 to 80, 1.1 to 70, 1.1 to 60, 1.1 to 50, 1.1 to 40, 1.1 to 30, 1.1 to 20, 1.1 to 10, 1.1 to 5, 1.1 to 2, 1.1 to 1.8, 1.1 to 1.5, 1.2 to 99, 1.2 to 90, 1.2 to 80, 1.2 to 70, 1.2 to 60, 1.2 to 50, 1.2 to 20, 1.2 to 10, 1.2 to 5, 1.2 to 2.5, 1.3 to 90, 1.3 to 80, 1.3 to 70, 1.3 to 50, 1.3 to 40, 1.3 to 30, 1.3 to 20, 1.3 to 10, 1.3 to 5, 1.3 to 1.5, 1.4 to 100, 1.4 to 95, 1.4 to 90, 1.4 to 80, 1.4 to 70, 1.4 to 60, 1.4 to 50, 1.4 to 30, 1.4 to 25, 1.4 to 20, 1.4 to 10, 1.4 to 5, 1.4 to 3, 1.4 to 2.5, 1.5 to 99, 1.5 to 95, 1.5 to 90, 1.5 to 80, 1.5 to 70, 1.5 to 60, 1.5 to 50, 1.5 to 50, 1.5 to 40, 1.5 to 30, 1.5 to 20, 1.5 to 10, 1.5 to 5, 1.5 to 2.5, 2 to 99, 2 to 90, 2 to 80, 2 to 70, 2 to 60, 2 to 50, 2 to 40, 2 to 35, 2 to 30, 2 to 20, 2 to 10, 2 to 5, 2 to 4, 2 to 2.5, 3 to 99, 3 to 90, 3 to 80, 3 to 70, 3 to 60, 3 to 50, 3 to 40, 3 to 30, 3 to 25, 3 to 20, 3 to 10, 4 to 99, 4 to 80, 4 to 70, 4 to 60, 4 to 50, 4 to 55, 4 to 25, 4 to 20, 4 to 15, 4 to 10, 5 to 100, 5 to 80, 5 to 50, 5 to 30, 5 to 20, 5 to 10, 5.5 to 9, 5.5 to 7, 10 to 90, 10 to 50, 10 to 20, 20 to 100, 20 to 50, 25 to 40, 20 to 35, 30 to 100, 30 to 50, 40 to 100, 40 to 70, 40 to 60, 40 to 50, 50 to 100, 50 to 90, 50 to 80, 50 to 70, 55 to 65, 60 to 80, 75 to 90, 75 to 100, 80 to 90, 80 to 85, 85 to 100.
65. Also provided for herein are immune cells, e.g. human immune cells, such as human T cells, treated with isolated viable mitochondria or comprising exogenous isolated viable mitochondria according to any one of the preceding items for use in a method of treating a subject in need thereof comprising administering to the subject the immune cells, or population of immune cells of any one of the preceding items.
66. The present disclosure further provides immune cells, e.g. human immune cells, such as human T cells, treated with isolated viable mitochondria or comprising exogenous isolated viable mitochondria according to any one of the preceding items for use in the treatment of cancer, infectious, inflammatory or autoimmune disease.
67. Also provided for herein is a composition, e.g. a pharmaceutical composition, according to the compositions, such as compositions comprising immune cell treated with isolated viable mitochondria or immune cells comprising exogenous viable mitochondria, of any one of the preceding items in an amount effective for use in the treatment of cancer, infectious, inflammatory or autoimmune disease.
68. In some aspects, the immune cells or population of immune cells treated with isolated viable mitochondria or comprising exogenous isolated viable mitochondria according to any one of the preceding items are formulated in a pharmaceutical composition in an amount effective for use in a method of treatment of cancer in a human subject in need thereof.
69. In some aspects, the immune cells or population of immune cells treated with isolated viable mitochondria or comprising exogenous viable mitochondria according to any one of the preceding items are formulated in a pharmaceutical composition in an amount effective for use in a method of treatment of autoimmune diseases in a human subject in need thereof. The autoimmune diseases include, but are not limited to, multiple sclerosis, diabetes, irritable bowel syndrome, Celiac disease, Crohn's disease, lupus, psoriasis, rheumatoid arthritis.
70. In some aspects, the immune cells or population of immune cells treated with isolated viable mitochondria or comprising exogenous mitochondria according to any one of the preceding items are formulated in a pharmaceutical composition in an amount effective for use in a method of treatment of inflammatory diseases in a human subject in need thereof.
71. In some aspects, the immune cells or population of immune cells treated with isolated viable mitochondria or comprising exogenous mitochondria according to any one of the preceding items are formulated in a pharmaceutical composition in an amount effective for use in a method of treatment of graft vs host diseases (GvHD) in a human subject in need thereof.
72. In some aspects, the immune cells or population of immune cells, e.g. anti-tumor cells, e.g. CAR-T cells, treated with isolated viable mitochondria or comprising exogenous mitochondria according to any one of the preceding items are formulated in a pharmaceutical composition in an amount effective for use in killing tumor cells, such as in killing tumor cells more effectively and/or for longer than equivalent immune cells or equivalent population of immune cells not treated with isolated viable mitochondria or lacking exogenous mitochondria.
73. In some aspects, the immune cells or population of immune cells treated with isolated viable mitochondria or comprising mitochondria according to any one of the preceding items are formulated in a pharmaceutical composition in an amount effective for use in autoimmune disease, e.g. in amount effective for use in lessening or preventing an aberrant immune response. In particular, the aberrant immune responses of the immune cells or population of immune cells treated with isolated viable or comprising exogenous isolated mitochondria according to any one of the preceding items, such as in the case of autoimmune diseases, are milder (lesser) and/or completely absent when compared to the responses of equivalent immune cells or equivalent population of immune cells not treated with isolated viable mitochondria or lacking exogenous mitochondria.
74. In some aspects, the immune cells or population of immune cells treated with isolated viable mitochondria or comprising exogenous mitochondria according to any one of the preceding items are formulated in a pharmaceutical composition and are transplanted in the subject in need of treatment through an autologous cell transplantation or allogeneic cell transplantation in an amount effective to treat cancer in a human subject in need thereof. In some aspects, the allogeneic cell transplantation comprises: (a) obtaining a sample of viable blood from a donor; (b) separating immune cells from the blood sample obtained in step (a); (c) transducing the immune cells with one or more exogenous polynucleotides encoding CARs or artificial TCR subunits; (d) optionally, contacting the immune cells with a small molecule; and (e) administering the modified immune cells into a subject in need thereof.
75. The present disclosure further provides immune cells or population of immune cells, e.g human immune cells, to be co-administered with a pharmaceutical composition comprising the isolated viable mitochondria formulated in a pharmaceutically acceptable carrier according to any one of the preceding items, in an amount effective for use in the treatment of cancer, infectious, inflammatory or autoimmune disease in a subject in need thereof. The co-administration of the pharmaceutical composition comprising isolated viable mitochondria may be prior to, simultaneously, or after the administration of the immune cell. In some aspects, the pharmaceutical composition is co-administered with the immune cells by intravenous infusion into the subject in need thereof. In some aspects, the pharmaceutical composition is co-administered with the immune cells via intratumoral injection. In some aspects, the pharmaceutical composition is co-administered with the immune cells via intraorgan injection, or through organ-specific vasculature. In some aspects, the subject has a cancer selected from the group: acute lymphoblastic leukemia (ALL), acute myeloid leukemia (AML), alveolar rhabdomyosarcoma, bladder cancer (e.g., bladder carcinoma), bone cancer, brain cancer (e.g., glioblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin's lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

### T Cells

In one embodiment, the immune cell comprising or enhanced by exogenous mitochondria is a T cell (also referred to as T lymphocytes), which belongs to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocyte types such as B cells and Natural Killer (NK) cells by the presence of cell surface proteins known as T cell receptors (TCR) that recognize antigens presented on the surface of cells. During a typical immune response, binding of these antigens to the T cell receptor, in the context of MHC antigen presentation, initiates intracellular changes leading to T cell activation.

T cells are divided into two groups by T cell receptors (TCRs), αβT cells and TST cells. αβT cells, with TCR2, mainly mediate cell immunity and immune-regulation while γδT cells, with TCR1, play important functions in wound healing, removing distressed or transformed epithelial cells and subduing excessive inflammation besides maintaining immune homeostasis in the local microenvironment. αβT cells and TST cells play different roles in autoimmune diseases, tumors and vascular diseases. αβT cells consist of 65-75% of peripheral blood mononuclear cells (PBMC) while γδT cells account for less than 10%. They express different surface markers of CD4 and CD8, e.g., 60% αβT cells are CD4 positive, 30% CD8 positive, and both positive less than 1% in αβT cells.

The term “activated T cells” as used herein, refers to T cells that have been stimulated to produce an immune response (e.g., clonal expansion of activated T cells) by recognition of an antigenic determinant, such as, for example, presented in the context of a Class I or Class II major histocompatibility (MHC) marker. T cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T cells (e.g., cells positive for CD3, CD4, or CD8 expression are referred to as CD3*, CD4⁺ or CD8⁺). CD3 and CD4 proteins are cell surface receptors or co-receptors that may be directly and/or indirectly involved in signal transduction in T cells.

In some embodiments, the immune cell comprising and/or enhanced by exogenous mitochondria comprises a CAR-T cell population. In some embodiments, the CAR-T cell population is selected, or enriched, or purified, to comprise at least 20%, 30%, 40%, 50%, 55%, 60%, 65%, 70%, 75%, 80%, 85%, 90%, 95%, 96%, 97%, 98% or 99%, for example, of a cell type that expresses a certain marker, receptor, or cell surface glycoprotein, such as, for example, CD8, CD4, CD3, CD34.

In some embodiments, the CAR-T cell population include CD4⁺ and CD8⁺ T cells. In some embodiments the CAR-T cell population is enriched to comprise at least 20%, 30%, 40%, 50%, 60%, 70%, 75%, 80%, 85%, 90%, 95%, 96%, 97%, 98% or 99% CD8⁺ T cells. In some embodiments the CAR-T cell population is enriched to comprise at least 80% CD8⁺ T cells. In some embodiments the CAR-T cell population is enriched to comprise at least 90% CD8⁺ T cells. Thus, in some embodiments, there are more genetically modified CD8⁺ T cells than genetically modified CD4⁺ T cells in the composition i.e., the ratio of CD4⁺ cells to CD8⁺ cells is less than 1, e.g., less than 0.9, less than 0.8, less than 0.7, less than 0.6, or less than 0.5.

### Enriched Immune Cell Populations

In some embodiments, enriched cell populations comprising or enhanced by exogenous mitochondria are provided, where the enriched cell population has been selected to comprise specified ratios or percentages of one or more cell type. By “cell population” or “modified cell population” is meant a group of cells, such as more than two cells. The cell population may be homogenous, comprising the same type of cell, or each comprising the same marker, or it may be heterogeneous. In some examples, the cell population is derived from a sample obtained from a subject and comprises cells prepared from, for example, bone marrow, umbilical cord blood, peripheral blood, or any tissue. In some examples, the cell population has been contacted with a nucleic acid, wherein the nucleic acid comprises a heterologous polynucleotide, such as, for example, a polynucleotide that encodes a chimeric antigen receptor, an inducible chimeric pro-apoptotic polypeptide, or a costimulatory polypeptide, such as, for example, a chimeric myeloid differentiation primary response 88 (MyD88) or truncated MyD88 and CD40 polypeptide. In some examples, the cell population and modified cell population are progeny of the original cells that have been contacted with the nucleic acid that comprises the heterologous polynucleotide. A cell population may be selected, or enriched, or purified, to comprise at least 20%, 30%, 40%, 50%, 55%, 60%, 65%, 70%, 75%, 80%, 85%, 90%, 95%, 96%, 97%, 98% or 99%, for example, of a cell type that expresses a certain marker, receptor, or cell surface glycoprotein, such as, for example, CD8, CD4, CD3, CD34.

Collecting T Lymphocytes from Patient's Resected Tumor and Enrichment of TIL Cells

T cells, such as TILs enhanced by exogenous mitochondria can be derived from a cancer patient. TILs are obtained from a resected tumor and expanded in vitro. Depending on the method applied, the isolation of the TILs leads to the re-infusion of “selected” or “young” TILs. Briefly, the resected tumors are processed, such as by enzymatic digestion, and the TILs are expanded and cultured in high dose of IL-2. An appropriate number of cells has to be obtained for re-infusion with autologous TILs. The “selected” TILs are tested for cytokine production upon tumor cell recognition, whereas the tumor reactivity of “young” TILs is not assessed. In general, “selected” TILs need up to 36 days from culture to tumor reactivity assessment before being re-introduced to the cancer patient. Of note, the expansion process of “young” TILs requires only between 10 to 22 days, while displaying comparable clinical responses compared to “selected” TILs. According to the present disclosure, TILs transplanted with exogenous mitochondria can be resected from any tumor and any protocol for expansion, re-infusion may be applied.

The selection, enrichment, or purification of a cell type in the modified cell population may be achieved by any suitable method. In some embodiments, the proportions of CD8⁺ and CD4⁺ T cells may be determined by flow cytometry. In some examples, a MACs column may be used. In some examples, the modified cell population is frozen and defrosted before administration to the subject, and the viable cells are tested for the percentage or ratio of a certain cell type before administration to the subject.

In some embodiments, the cell population is selected, or enriched, or purified, to comprise at least 20%, 30%, 40%, 50%, 55%, 60%, 65%, 70%, 75%, 80%, 85%, 90%, 95, 96, 97, 98, or 99% of CD8⁺ or CD4⁺ T cells.

According to the present disclosure, mitochondria preparations comprising e.g., autologous mitochondria, allogeneic mitochondria, xenogeneic mitochondria, encapsulated mitochondria or autogenous mitochondria with appropriate genetic modification may be delivered to enriched T cells.

Collecting T Lymphocytes from Patient's Blood and Enrichment of T Cells

T cells, such as T cells enhanced by exogenous mitochondria and/or engineered to express a CAR, can be derived from any healthy donor. The donor will generally be an adult (at least 18 years old) but children are also suitable as T cell donors (Styczynski, 2018, “Young child as a donor of cells for transplantation and lymphocyte based therapies”, Transfus Apher Sci 57:323-30). An example of a suitable process for obtaining T cells from a donor is described in (Di Stasi et al., 2011, “Inducible apoptosis as a safety switch for adoptive cell therapy”, N Engl J Med 365:1673-83). In general, T cells are obtained from a donor, subjected to genetic modification and selection, and can then be administered to recipient subjects. A useful source of T cells is the donor's peripheral blood. Peripheral blood samples will generally be subjected to leukapheresis to provide a sample enriched for white blood cells. This enriched sample (also known as a “leukopak”) can be composed of a variety of blood cells including monocytes, lymphocytes, platelets, plasma, and red cells. Elimination of contaminants, like red blood cells, platelets, monocytes, and tumor cells, requires a multi-pronged approach generally required using methods known in the art. A leukopak typically contains a higher concentration of cells as compared to venipuncture or buffy coat products.

Patients with relapsed cancer may have low T-cell counts, thus making it difficult to collect sufficient autologous T cells. This issue can be overcome by methods known in the art, such as by using allogeneic T lymphocytes collected from healthy donors.

The selection, enrichment, or purification of a cell type in the modified cell population may be achieved by any suitable method. In some embodiments, the proportions of CD8⁺ and CD4⁺ T cells may be determined by flow cytometry. In some examples, a MACs column may be used. In some examples, the modified cell population is frozen and defrosted before administration to the subject, and the viable cells are tested for the percentage or ratio of a certain cell type before administration to the subject. Whereas the ratio of CD4⁺ cells to CD8⁺ cells in a leukopak is typically above 2, in some embodiments the ratio of CD4⁺ cells to CD8⁺ cells in a composition of the invention is less than 2, e.g., less than 1.5. In some embodiments, there are more CD8⁺ T cells than CD4⁺ T cells in the composition, i.e., the ratio of CD4⁺ cells to CD8⁺ cells is less than 1 e.g. less than 0.9, less than 0.8, less than 0.7, less than 0.6, or less than 0.5. Thus, the overall procedure starting from donor cells and producing T cells is designed to enrich for CD8⁺ cells T cells relative to CD4⁺ T cells. In some embodiments, 60% or more of the T cells are CD8⁺ T cells, and in some embodiments, 65% or more of the T cells are CD8⁺ T cells. Within the population of CD3⁺ T cells, in some embodiments, the percent of CD8⁺ T cells is between 55-75%, for example, from 55%-65%, from 55%-70%, from 56-71%, from 63-73%, from 60-70%, from 59%-74%, from 65-71% or from 65-75%. In some embodiments, a cell population is provided that is selected, or enriched, or purified, to comprise a ratio of one cell type to another, such as, for example, a ratio of CD8⁺ to CD4⁺ T cells of, for example, 3:2, 7:3, 4:1, 9:1, 19:1, or 39:1 or more. In some embodiments, the modified cell population is selected, or enriched, or purified, to comprise at least 20%, 30%, 40%, 50%, 55%, 60%, 65%, 70%, 75%, 80%, 85%, 90%, 95, 96, 97, 98, or 99%, CD8⁺ T cells. In some embodiments, the ratio of CD8⁺ to CD4⁺ T cells is 4-to-1, or 9-to-1 or greater.

In some embodiments, for a population of genetically modified CD3⁺ T cells comprising a costimulatory polypeptide as described herein, the percent of CD8⁺ T cells is between 55-75%, for example, from 55-65%, from 55-70%, from 56-71%, from 59-74%, from 63-73%, from 60-70%, from 60-75%, from 65-75%, or from 65-71%. In some embodiments, the ratio of CD8⁺ to CD4⁺ T cells is 3:2, 7:3, 4:1, 9:1, 19:1, or 39:1 or more. In some embodiments, the modified cell population comprising a costimulatory polypeptide is selected, or enriched, or purified, to comprise at least 20%, 30%, 40%, 50%, 60%, 70%, 75%, 80%, 85%, 90%, 95, 96, 97, 98, or 99%, CD8⁺ T cells. In some embodiments, the ratio of CD8⁺ to CD4⁺ T cells is 4-to-1, or 9-to-1 or greater. The costimulatory polypeptide can comprise one or more costimulatory signaling regions such as CD27, ICOS, RANK, TRANCE, CD28, 4-1BB, OX40, DAP10, MyD88, or CD40. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40. The costimulatory polypeptide can be inducible or constitutively activated.

In some embodiments, the invention provides compositions and methods comprising a CAR-T cell population comprising an inducible pro-apoptotic polypeptide where at least 80%, 85%, 90%, 95, 96, 97, 98, or 99%, are CD8⁺ T cells. In some embodiments, the modified cell population comprising an inducible pro-apoptotic polypeptide is at least 80% CD8⁺ T cells. In some embodiments, the modified cell population is at least comprising an inducible pro-apoptotic polypeptide 90% CD8⁺ T cells.

In some embodiments, the invention provides compositions and methods comprising a CAR-T cell population comprising a costimulatory polypeptide and an inducible pro-apoptotic polypeptide where at least 80%, 85%, 90%, 95, 96, 97, 98, or 99%, are CD8⁺ T cells. In some embodiments, the modified cell population comprising a costimulatory polypeptide and an inducible pro-apoptotic polypeptide is at least 80% CD8⁺ T cells. In some embodiments, the modified cell population comprising a costimulatory polypeptide and an inducible pro-apoptotic polypeptide is at least 90% CD8⁺ T cells.

According to the present disclosure, mitochondria preparations comprising e.g., autologous mitochondria, allogeneic mitochondria, xenogeneic mitochondria, encapsulated mitochondria or autogenous mitochondria with appropriate genetic modification may be delivered to enriched T cells before, concurrently with, or after genetic modification (e.g., introduction of the CAR gene) is performed.

### Mitochondria

The present invention is based, at least in part, on the discovery that isolated mitochondria can be delivered to (also referred to as transplanted into) cultured cells or a patient's tissue by adding them to a cell culture or by injecting them into the patient's tissue or blood vessels leading to the tissue, respectively (Cowan et al., 2017, “Transit and integration of extracellular mitochondria in human heart cells”, Sci Rep 7:17450; McCully et al., 2017, “Mitochondrial transplantation: From animal models to clinical use in humans”, Mitochondrion 34:127-34).

Mitochondria can be delivered ex vivo to cells of interest. Cells of interest include, but are not limited to, any of the immune cells described herein cultured cells, previously engineered immune cells (e.g., CAR T cells), or cells to be further engineered (e.g., to express a CAR or artificial TCR) and/or cultured (e.g., differentiated, activated, treated, or incubated). Mitochondria can be delivered ex vivo by liposome-mediated transfer using the synthetic liposomes, such as Lipofectin® (Shi et al., 2008. “Mitochondria transfer into fibroblasts: liposome-mediated transfer of labeled mitochondria into cultured cells”, Ethn. Dis. 18: S1-43). Mitochondria can be delivered ex vivo through co-incubation (i.e., co-culturing) of the cells, such as any of the immune cells described herein, with mitochondria over the period of 2-24 hours (Masuzawa et al., 2013, “Transplantation of autologously derived mitochondria protects the heart from ischemia-reperfusion injury”, Am J Physiol Heart Circ Physiol 304:H966-82). Without wishing to be bound by theory, transplanted mitochondria are internalized by an actin dependent pathway. Mitochondrial internalization, such as previously demonstrated in cardiomyocytes, can occur following a 1-hour co-incubation (Pacak et al., 2015, “Actin-dependent mitochondrial internalization in cardiomyocytes: evidence for rescue of mitochondrial function”, Biol Open 4:622-6).

Mitochondria can also be delivered into an organ or tissue by direct injection into the targeted area, or by delivery through the organ- or tissue-specific vasculature, such as the coronary artery of the subject, the pulmonary artery of the subject, the hepatic portal vein of the subject, the greater pancreatic artery of the subject, the renal artery of the subject, or the prostate artery of the subject. In the latter case, mitochondria are retained in the downstream organ or tissue. For example, when administered through the coronary arteries, mitochondria are almost exclusively delivered to the heart (Shin et al., 2019, “Myocardial Protection by Intracoronary Delivery of Mitochondria: Safety and Efficacy in the Ischemic Myocardium”, JACC: Basic to Translational Science Vol. 4, No. 8, 2019), while the mitochondria may be delivered into the lung through the pulmonary artery, or into the kidneys by delivery through the renal arteries. The direct injection of mitochondria allows for focal concentration of the injected mitochondria. The number of mitochondria used for injection may vary, depending on the size of the targeted organ or tissue as well as the intended use. The mitochondria may be suspended in homogenizing buffer and injected at various sites using e.g. a tuberculin syringe with a 28-32 gauge needle (Emani et al., 2017, “Autologous mitochondrial transplantation for dysfunction after ischemia-reperfusion injury”, J Thorac Cardiovasc Surg 154:286-9; McCully et al., 2017, “Mitochondrial transplantation: From animal models to clinical use in humans”, Mitochondrion 34:127-34).

Mitochondrial transplantation in vivo can be performed using either single or serial injections of either autologous or heterologous mitochondria, with no direct or indirect, acute or chronic alloreactivity, allorecognition, or damage-associated molecular pattern molecules (Ramirez-Barbieri et al., 2019, “Alloreactivity and allorecognition of syngeneic and allogeneic mitochondria”, Mitochondrion 46:103-15).

Without wishing to be bound by theory, viable, respiration competent mitochondria are taken up by both ischemic and non-ischemic tissue by endocytosis (Cowan et al., 2016, “Intracoronary Delivery of Mitochondria to the Ischemic Heart for Cardioprotection”, PLoS One 11:e0160889; Kesner et al., 2016, “Characteristics of Mitochondrial Transformation into Human Cells”, Sci Rep 6:26057; Cowan et al., 2017, “Transit and integration of extracellular mitochondria in human heart cells”, Sci Rep 7:17450).

Skilled practitioners can locally and/or generally distribute mitochondria to tissues and/or cells of a patient for a variety of purposes, using relatively simple medical procedures. Compared to some traditional therapeutic regimens that involve nanoparticles, it is further noted that mitochondria are not toxic and do not cause any substantial adverse immune or auto-immune response.

While not intending to be bound by any theory, it is believed that infused mitochondria extravasate through the capillary wall by first adhering to the endothelium. After they are injected or infused into an artery, mitochondria can cross the endothelium of the blood vessels and be taken up by tissue cells through an endosomal actin-dependent internalization process.

Mitochondrial transplantation in vivo can include co-administration of any of the cells of interest described herein together with the exogenous mitochondria (e.g. exogenous isolated viable mitochondria) provided herein. In some embodiments, exogenous mitochondria and cells of interest are co-administered to promote or enhance the desired therapeutic effect of the cells of interest to treat a disease in a patient. Cells of interest include, but are not limited to, any of the immune cells described herein, cultured cells, previously engineered immune cells (e.g., CAR T cells), or cells to be further engineered (e.g., to express a CAR or artificial TCR). In embodiments where exogenous mitochondria and the cells of interest are included in different pharmaceutical compositions, administration of the exogenous mitochondria can occur prior to, simultaneously with, or following, administration of the cells of interest. In some aspects, administration of exogenous mitochondria and cells of interest occur within about one month of each other. In some aspects, administration of exogenous mitochondria and cells of interest occur within about one week of each other. In some aspects, administration of exogenous mitochondria and the cells of interest occur within about five, four, three or two days of each other. In some aspects, administration of exogenous mitochondria and the cells of interest occur within about one day of each other. In some aspects, administration of exogenous mitochondria and cells of interest occur within about twelve hours of each other. In some aspects, administration of exogenous mitochondria and cells of interest occur within about six hours of each other. In some aspects, administration of exogenous mitochondria and cells of interest occur within about three hours of each other. In some aspects, administration of exogenous mitochondria and cells of interest occur within about two hours of each other. In some aspects, administration of exogenous mitochondria and cells of interest occur within about one hour of each other. In some aspects, administration of exogenous mitochondria and cells of interest occur within about thirty minutes of each other. In some aspects, administration of exogenous mitochondria and cells of interest occur within about fifteen minutes of each other. In some aspects, administration of exogenous mitochondria and the cells of interest occur within minutes of each other. In some aspects, co-administration of exogenous mitochondria and cells of interest include repeated administration of exogenous mitochondria and/or cells of interest.

### Isolating Mitochondria

Mitochondria for use in the presently described methods can be isolated or provided from any source, e.g., isolated from cultured cells or tissues. Exemplary cells include, but are not limited to, muscle tissue cells, cardiac fibroblasts, HeLa cells, prostate cancer cells, yeast, among others, and any mixture thereof. Exemplary tissues include, but are not limited to, liver tissue, skeletal muscle, heart, brain, and adipose tissue. Mitochondria can be isolated from cells or tissues (e.g., biopsy material) of an autogenous source, an allogeneic source, and/or a xenogeneic source. In some instances, mitochondria are isolated from cells with a genetic modification, e.g., cells with modified mtDNA or modified nuclear DNA.

Mitochondria can be isolated from cells or tissues by any means known to those of skill in the art. In one example, tissue samples or cell samples are collected and then homogenized. Following homogenization, mitochondria are isolated by repetitive centrifugation (Kesner et al., 2016, “Characteristics of Mitochondrial Transformation into Human Cells”, Sci Rep 6:26057). Alternatively, the cell homogenate can be filtered through nylon mesh filters. Typical methods of isolating mitochondria are described, for example, in McCully J D, Cowan D B, Pacak C A, Toumpoulis I K, Dayalan H and Levitsky S, “Injection of isolated mitochondria during early reperfusion for cardioprotection”, Am J Physiol 296, H94-H105. PMC2637784 (2009); Frezza, C., Cipolat, S., & Scorrano, L, “Organelle isolation: functional mitochondria from mouse liver, muscle and cultured fibroblasts”, Nature protocols, 2(2), 287-295 (2007); and a PCT application entitled “Products and Methods to Isolate Mitochondia” (PCT/US2015/035584; WO 2015192020); each of which is incorporated by reference.

Mitochondria, such as those used in therapy or included in a pharmaceutical composition, can be isolated from cells or tissues of an autogenous source, an allogeneic source, or a xenogeneic source. In some instances, mitochondria are collected from cultured cells or tissues of a subject, and these mitochondria are administered back to the same subject (autologous). In some other cases, mitochondria are collected from cultured cells (e.g., human cardiac fibroblasts) or tissues of a second subject, and these mitochondria are administered to a first subject (allogeneic). In some cases, mitochondria are collected from cultured cells or tissues from a different species (e.g., mice, swine, and yeast) (xenogeneic).

In certain embodiments of methods described herein, the mitochondria can have different sources, e.g., the exogenous mitochondria can be autologous, autogeneic, allogeneic, or xenogeneic. In certain embodiments the mitochondria have been freshly isolated (within 120 min after taking the tissue biopsy samples, preferably within 60 minutes, more preferably within 30 minutes). In some embodiments the mitochondria have been isolated and subsequently stored until use. In certain embodiments, the autogeneic mitochondria can have exogenous mtDNA. In some embodiments, the mitochondria are from a subject's first-degree relative. In some embodiments, the mitochondria have been encapsulated.

In some embodiments, the described methods include the step of collecting the isolated mitochondria from cells prior to administration. The isolated mitochondria can be transplanted into cells of interest, e.g., any of the immune effector cells described herein, or administered to the subject in conjunction with the treatment with cells of interest.

### Engineering Expression Constructs

In some embodiments, the immune cell comprising or enhanced by exogenous mitochondria is engineered, such as engineered to express a CAR as used herein, the term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

In some embodiments, a nucleic acid construct, e.g., any of the chimeric antigen receptors described herein, is contained within a viral vector. In certain embodiments, the viral vector is a retroviral vector. In certain embodiments, the viral vector is an adenoviral vector or a lentiviral vector. It is understood that in some embodiments, a cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, described herein.

As used herein, the term “gene” is defined as a functional protein-, polypeptide-, or peptide encoding unit. As will be understood, this functional term includes genomic sequences, cDNA sequences, and smaller engineered gene segments that express, or are adapted to express, proteins, polypeptides, domains, peptides, fusion proteins and/or mutants.

Promoters, and other regulatory elements, are selected such that they are functional in the desired cells or tissue. In addition, this list of promoters should not be construed to be exhaustive or limiting; other promoters that are used in conjunction with the promoters and methods disclosed herein.

Expression constructs, such as CAR genes, can be incorporated randomly into the genome, such as through viral mediated integration, or purposely integrated into the specific sites of an immune cell genome, such as a T-cell genome, including but not limited to CCR5 and AAVS1 loci, or into the T-cell receptor a constant (TRAC) locus. Targeted integration can use gene-editing tools such as nuclease-meditated genome editing systems, including the clustered regularly interspaced short palindromic repeats (CRISPR/Cas9) system, zinc-finger nucleases (ZFNs), and transcription activator-like effector nucleases (TALENs) (Liu et al., 2019, “Building Potent Chimeric Antigen Receptor T Cells With CRISPR Genome Editing”, Front Immunol 10:456).

### Costimulation

In some embodiments, the immune cell comprising or enhanced by exogenous mitochondria is an immune cell engineered to express a CAR, such as a CAR-T cell, comprising a costimulatory polypeptide. In some embodiments, the immune cell comprising or enhanced by exogenous mitochondria is a CAR-T cell comprising a costimulatory polypeptide. The CARs can be engineered to include a costimulation domain, such as those derived from the cytoplasmic portion of T cell costimulatory molecules, including, but not limited to, CD28, 4-1BB, OX40, ICOS and DAP10 (see, e.g., Carpenito et al. (2009) Proc Natl Acad Sci U.S.A. 106:3360-3365; Finney et al. (1998) J Immunol 161:2791-2797; Hombach et al. J Immunol 167:6123-6131; Maher et al. (2002) Nat Biotechnol 20:70-75; Imai et al. (2004) Leukemia 18:676-684; Wang et al. (2007) Hum Gene Ther 18:712-725; Zhao et al. (2009) J Immunol 183:5563-5574; Milone et al. (2009) Mol Ther 17: 1453-1464; Yvon et al. (2009) Clin Cancer Res 15:5852-5860), which allow CAR-T cells to receive appropriate costimulation upon engagement of the target antigen.

The costimulatory polypeptide of the present invention can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40 or, for example, the cytoplasmic regions thereof. The costimulatory polypeptide can comprise one or more suitable costimulatory signaling regions that activate the signaling pathways activated by CD27, ICOS, RANK, TRANCE, CD28, 4-1BB, OX40, DAP10, MyD88, or CD40. Costimulatory polypeptides include any molecule or polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulatory polypeptide or costimulatory polypeptide cytoplasmic region may be expressed in the modified T cells discussed herein.

In some embodiments, the inducible chimeric signaling polypeptide comprises two costimulatory polypeptide cytoplasmic signaling regions, such as, for example, 4-1BB and CD28, or one, or two or more costimulatory polypeptide cytoplasmic signaling regions selected from the group consisting of CD27, ICOS, RANK, TRANCE, CD28, 4-1BB, OX40, DAP10.

### Vectors

In some embodiments, the population of immune cells comprising or enhanced by exogenous mitochondria (e.g., as autologous, allogeneic mitochondria, xenogeneic mitochondria, encapsulated mitochondria or autogenous mitochondria with appropriate genetic modification) comprises a CAR or artificial TCR subunit produced from a DNA, double-stranded RNA, single-stranded mRNA, or circular RNA vector. It is understood that the vectors provided herein may be modified using methods known in the art to vary the position or order of the regions, to substitute one region for another A vector can encode antigen-binding domains, e.g., as part of a CAR construct, specific for one or more target antigens, such as, for example, BCMA, CD123, CD20, CD22, CD30, CD33, EGFR, EGFRvIII, GD2, Her2, Mesothelin, MUC1, MUC16, NKG2D, NY-ESO-1, PRAME, PSCA, PSMA, ROR1, etc. The vector may also be modified with appropriate substitutions of each polypeptide region, as discussed herein.

A vector can encode co-stimulatory polypeptide cytoplasmic signaling regions, e.g., as part of a CAR construct, comprising one, or two or more co-stimulatory polypeptide cytoplasmic signaling regions such as, for example, those selected from the group consisting of CD27, CD28, 4-1BB, OX40, ICOS, RANK, TRANCE, and DAP10. A vector can encode a linker, e.g., as part of a CAR construct, such as a linker between the CAR polypeptide and the co-stimulatory polypeptide. Engineered immune cells, such as T cells (e.g., CAR T cells), of the invention may express a safety switch, also known as an inducible suicide gene or suicide switch, which can be used to eradicate the engineered immune cells in vivo if desired e.g. if graft versus host disease (GVHD) develops. In some examples, engineered immune cells that express a chimeric antigen receptor are provided to the patient that trigger an adverse event, such as on-target off-tumor toxicity. In some therapeutic instances, a patient might experience some negative symptoms during therapy using CAR-modified cells. In some cases, these therapies have led to adverse events due, in part, to non-specific attacks on healthy tissue. In some examples, the therapeutic engineered immune cells may no longer be needed, or the therapy is intended for a specified amount of time, for example, the therapeutic engineered immune cells may work to decrease the tumor cell, or tumor size, and may no longer be needed. Therefore, in some embodiments are provided nucleic acids, cells, and methods wherein the engineered immune cell also expresses a safety switch, such as an inducible caspase-9 polypeptide. Other suicide switch systems known in the art include, but are not limited to, (a) herpes simplex virus (HSV)-tk which turns the nontoxic prodrug ganciclovir (GCV) into GCV-triphosphate, leading to cell death by halting DNA replication, (b) iCasp9 can bind to the small molecule AP1903 and result in dimerization, which activates the intrinsic apoptotic pathway, and (c) Targetable surface antigen expressed in the transduced iNKT cells (e.g., CD20 and truncated EGFR), allowing eliminating the modified cells efficiently through complement/antibody-dependent cellular cytotoxicity (CDC/ADCC) after administration of the associated monoclonal antibody. If there is a need, for example, to reduce the number of engineered immune cells, an inducible ligand may be administered to the patient, thereby inducing apoptosis of the engineered immune cells. These switches respond to a trigger, such as a pharmacological agent, which is supplied when it is desired to eradicate the engineered immune cells, and which leads to cell death (e.g., by triggering necrosis or apoptosis). These agents can lead to expression of a toxic gene product, but a more rapid response can be obtained if the engineered immune cells already express a protein, which is switched into a toxic form in response to the agent.

### Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually, the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g., CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, β-gal or chloramphenicol acetyltransferase (CAT).

### Linker Polypeptides

Linker polypeptides include, for example, cleavable and non-cleavable linker polypeptides. Non-cleavable polypeptides may include, for example, any polypeptide that may be operably linked between the costimulatory polypeptide cytoplasmic signaling region and ITAM portion of the chimeric antigen receptor (e.g., CD3ζ). Linker polypeptides include those for example, consisting of about 2 to about 30 amino acids, (e.g., furin cleavage site or glycine-serine linker, such as (GGGGS)ₙ). In some embodiments, the linker polypeptide consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the linker polypeptide consists of about 18 to 22 amino acids. In some embodiments, the linker polypeptide consists of 20 amino acids. In some embodiments, cleavable linkers include linkers that are cleaved by an enzyme exogenous to the modified cells in the population, for example, an enzyme encoded by a polynucleotide that is introduced into the cells by transfection or transduction, either at the same time or a different time as the polynucleotide that encodes the linker. In some embodiments, cleavable linkers include linkers that are cleaved by an enzyme endogenous to the modified cells in the population, including, for example, enzymes that are naturally expressed in the cell, and enzymes encoded by polynucleotides native to the cell, such as, for example, lysozyme

### Therapeutic Applications

The immune cells enhanced with exogenous mitochondria, e.g. exogenous isolated viable mitochondria, provided herein (such as immune cells into which autologous mitochondria, allogeneic mitochondria, xenogeneic mitochondria, encapsulated mitochondria or mitochondria with genetic modification were transplanted) may be useful for the treatment of any disease or condition involving a target. If the application discloses a general application of immune cells (not binder-specific) then can use “tumor associated antigen” (“TAA”) as the target cell molecule. In some embodiments, the disease or condition is a disease or condition that can benefit from treatment with adoptive cell therapy. In some embodiments, the disease or condition is a tumor. In some embodiments, the disease or condition is a cell proliferative disorder. In some embodiments, the disease or condition is a cancer. In some embodiments, the disease or condition is a viral infection. In some embodiments, the disease or condition is an autoimmune disease.

In some embodiments, provided herein is a method of treating a disease or condition in a subject in need thereof by administering to the subject an effective amount of an immune cell enhanced with exogenous mitochondria provided herein, e.g., immune cells previously transplanted with exogenous mitochondria ex vivo. In some embodiments, provided herein is a method of treating a disease or condition in a subject in need thereof by co-administering to the subject an effective amount of an immune cell together with exogenous mitochondria provided herein to the subject. In some aspects, the disease or condition is a cancer. In some aspects, the disease or condition is a viral infection. In some embodiments, the disease or condition is an autoimmune disease.

Any suitable cancer may be treated with the immune cells enhanced with exogenous mitochondria provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin's lymphoma (HL), hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin's lymphoma (NHL), non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer (SCLC), small intestine cancer, soft tissue sarcoma, spinal cord tumor, stomach cancer, T cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

### Combination Therapies

In some embodiments, the immune cells, such as T cells or CAR T cells, enhanced with exogenous mitochondria provided herein are administered with at least one additional therapeutic agent. Immune cells enhanced with exogenous mitochondria can include immune cells previously transplanted with exogenous mitochondria ex vivo, or immune cells co-administered with exogenous mitochondria such that exogenous mitochondria are transplanted into immune cells in vivo. Any suitable additional therapeutic agent may be administered with an immune cell enhanced with exogenous mitochondria provided herein. In some aspects, the additional therapeutic agent is selected from radiation, a cytotoxic agent, a chemotherapeutic agent, a cytostatic agent, an anti-hormonal agent, an EGFR inhibitor, an immunostimulatory agent, an anti-angiogenic agent, a checkpoint blockade agent, and combinations thereof.

In some embodiments, the additional therapeutic agent comprises an immunostimulatory agent.

In some embodiments, the immunostimulatory agent is an agent that blocks signaling of an inhibitory receptor of an immune cell, or a ligand thereof. In some aspects, the inhibitory receptor or ligand is selected from cytotoxic T-lymphocyte-associated protein 4 (CTLA-4, also known as CD152), programmed cell death protein 1 (also PD-1 or CD279), programmed death ligand 1 (also PD-L1 or CD274), transforming growth factor beta (TGFβ), lymphocyte-activation gene 3 (LAG-3, also CD223), Tim-3 (hepatitis A virus cellular receptor 2 or HAVCR2 or CD366), neuritin, B- and T-lymphocyte attenuator (also BTLA or CD272), killer cell immunoglobulin-like receptors (KIRs), and combinations thereof. In some aspects, the agent is selected from an anti-PD-1 antibody (e.g., pembrolizumab or nivolumab), and anti-PD-L1 antibody (e.g., atezolizumab), an anti-CTLA-4 antibody (e.g., ipilimumab), an anti-TIM3 antibody, carcinoembryonic antigen-related cell adhesion molecule 1 (CECAM-1, also CD66a) and 5 (CEACAM-5, also CD66e), vset immunoregulatory receptor (also VISR or VISTA), leukocyte-associated immunoglobulin-like receptor 1 (also LAIR1 or CD305), CD160, natural killer cell receptor 2B4 (also CD244 or SLAMF4), and combinations thereof. In some aspects, the agent is pembrolizumab. In some aspects, the agent is nivolumab. In some aspects, the agent is atezolizumab.

In some embodiments, the additional therapeutic agent is an agent that inhibits the interaction between PD-1 and PD-L1. In some aspects, the additional therapeutic agent that inhibits the interaction between PD-1 and PD-L1 is selected from an antibody, a peptidomimetic and a small molecule. In some aspects, the additional therapeutic agent that inhibits the interaction between PD-1 and PD-L1 is selected from pembrolizumab (Keytruda™), nivolumab (Opdivo™), atezolizumab (Tecentriq™), avelumab (Bavencio™), pidilizumab, durvalumab, BMS-936559, sulfamonomethoxine 1, and sulfamethizole 2. In some embodiments, the additional therapeutic agent that inhibits the interaction between PD-1 and PD-L1 is any therapeutic known in the art to have such activity, for example as described in Weinmann et al. (Weinmann, 2016, “Corrigendum: Cancer Immunotherapy: Selected Targets and Small-Molecule Modulators”, Chem Med Chem 11:1576), incorporated by reference in its entirety. In some embodiments, the agent that inhibits the interaction between PD-1 and PD-L1 is formulated in the same pharmaceutical composition an antibody provided herein. In some embodiments, the agent that inhibits the interaction between PD-1 and PD-L1 is formulated in a different pharmaceutical composition from an antibody provided herein. In some embodiments, the agent that inhibits the interaction between PD-1 and PD-L1 is administered prior to administration of an antibody provided herein. In some embodiments, the agent that inhibits the interaction between PD-1 and PD-L1 is administered after administration of an antibody provided herein. In some embodiments, the agent that inhibits the interaction between PD-1 and PD-L1 is administered contemporaneously with an antibody provided herein, but the agent and antibody are administered in separate pharmaceutical compositions.

In some embodiments, the immunostimulatory agent is an agonist of a co-stimulatory receptor of an immune cell. In some aspects, the co-stimulatory receptor is selected from GITR, OX40, ICOS, LAG-2, CD27, CD28, 4-1BB, CD40, STING, a toll-like receptor, RIG-1, and a NOD-like receptor. In some embodiments, the agonist is an antibody.

In some embodiments, the immunostimulatory agent modulates the activity of arginase, indoleamine-2 3-dioxygenase, or the adenosine A2A receptor.

In some embodiments, the immunostimulatory agent is a cytokine. In some aspects, the cytokine is selected from IL-2, IL-5, IL-7, IL-12, IL-15, IL-21, and combinations thereof. In some aspects, the cytokine is IL-2.

In some embodiments, the immunostimulatory agent is an oncolytic virus. In some aspects, the oncolytic virus is selected from a herpes simplex virus, a vesicular stomatitis virus, an adenovirus, a Newcastle disease virus (NDV), a vaccinia virus, and a maraba virus.

Further examples of additional therapeutic agents include a taxane (e.g., paclitaxel or docetaxel); a platinum agent (e.g., carboplatin, oxaliplatin, and/or cisplatin); a topoisomerase inhibitor (e.g., irinotecan, topotecan, etoposide, and/or mitoxantrone); folinic acid (e.g., leucovorin); or a nucleoside metabolic inhibitor (e.g., fluorouracil, capecitabine, and/or gemcitabine). In some embodiments, the additional therapeutic agent is folinic acid, 5-fluorouracil, and/or oxaliplatin. In some embodiments, the additional therapeutic agent is 5-fluorouracil and irinotecan. In some embodiments, the additional therapeutic agent is a taxane and a platinum agent. In some embodiments, the additional therapeutic agent is paclitaxel and carboplatin. In some embodiments, the additional therapeutic agent is pemetrexed. In some embodiments, the additional therapeutic agent is a targeted therapeutic such as an EGFR, RAF or MEK-targeted agent.

The additional therapeutic agent may be administered by any suitable means. In some embodiments, a medicament provided herein, and the additional therapeutic agent are included in the same pharmaceutical composition. In some embodiments, an antibody provided herein, and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some aspects, administration of an antibody provided herein, and the additional therapeutic agent occur within about one month of each other. In some aspects, administration of an antibody provided herein, and the additional therapeutic agent occur within about one week of each other. In some aspects, administration of an antibody provided herein, and the additional therapeutic agent occur within about one day of each other. In some aspects, administration of an antibody provided herein, and the additional therapeutic agent occur within about twelve hours of each other. In some aspects, administration of an antibody provided herein, and the additional therapeutic agent occur within about one hour of each other.

### Methods of Use

The present specification provides methods to deliver isolated mitochondria or pharmaceutical compositions of isolated mitochondria ex vivo to the cells of a patient or allogeneic donor and/or in vivo to tissues of a patient. Without wishing to be bound by theory, mitochondria are taken up by tissue cells or cultured cells through an actin-dependent endocytosis, thereby providing a way to deliver the pharmaceutic composition directly into the cells. In a non-limiting illustrative example, mitochondria are transplanted into the target immune cells by e.g., co-incubation of mitochondria (100 μg/well) with the cells (10⁶/well) in culture medium over the period of 2-24 hours. One skilled in the art can recognize the dosage of mitochondria administered to immune cells ex vivo or to tissues of a patient in vivo may be varied based on the intended outcome in terms of enhancing the target immune cell or cells, such as optimization of viability, survival, endurance, self-renewal capacity and/or selection. In the ex vivo delivery of mitochondria to immune cells, e.g., through co-incubation, the dosage of mitochondria may be between 0.0001 ng of mitochondria per target-cell and 2.5 ng of mitochondria per target cell. In delivery of mitochondria in vivo, to the tissue of a patient, between 1 mitochondrion and 10⁷ Mitochondria per 1 mL may be delivered.

The present disclosure contemplates a composition comprising enhanced immune cells (αβT cells, TST cells, memory immune cells (e.g. central memory CD8 T cell, effector memory CD8 T cells, or memory-like T cells), Treg cells (e.g. Treg CD4 T cells), CAR-T cells, etc.), wherein the cells comprise or are enhanced by exogenous mitochondria, which may be autologous mitochondria, allogeneic mitochondria, xenogeneic mitochondria, encapsulated mitochondria or autogenous mitochondria with genetic modification. These cells can be either any effector cells known in the art with anti-tumor activity or immunosuppressive immune cells able to prevent autoimmunity. Accordingly, the present specification provides methods to deliver immune cells comprising or enhanced by exogenous mitochondria, or pharmaceutical compositions of immune cells comprising or enhanced by exogenous mitochondria, to the cells and/or tissues of a patient or cells derived from an allogeneic donor. The immune cells comprising or enhanced by exogenous mitochondria can be used to treat a variety of diseases, including but not limited to various forms of cancer, tumors and autoimmune disease.

In some embodiments, preparation of CAR T cells can include the following steps:
1. Collecting T lymphocytes from patient's blood by leukapheresis.
2. Enrichment of T cells by density gradient centrifugation, elutriation, and immunomagnetic bead selection.
3. Gene modification using electroporation, retroviral/lentiviral transduction, or nuclease-meditated genome editing (e.g., introduction of CAR gene into the genome of the target cell).
4. Activation and expansion of CAR-T cells via polyclonal activation through artificial antigen presenting systems (anti-CD8/anti-CD28 immunomagnetic beads/LV-APCs) using methods known in the art.
   Consistency is generally achieved through standardization and validation of raw materials and protocols according to cGMPs (current good manufacturing practices).
5. Quality Assurance—testing for viability, phenotyping, gram staining, endotoxin, and bacterial, fungal, and mycoplasma contaminants pursuant to the FDA guidelines using methods known in the art.
6. Formulation and Administration—testing for clinically prescribed dosage and route of administration using methods known in the art.
   Therapeutic cell preservation, packaging, transport, receipt, and administration generally should maintain product stability and chain of custody.

In a particular embodiment, mitochondria preparations are delivered to immune cells (1) before, (2) concurrently with, or (3) after genetic modification (e.g., introduction of the CAR gene) is performed. In a particular embodiment, mitochondria preparations are delivered ex vivo to immune cells (1) before, (2) concurrently with, or (3) after ex vivo genetic modification (e.g., introduction of the CAR gene) is performed, such as in methods including ex vivo genetic modification. In a particular embodiment, mitochondria preparations are delivered ex vivo to immune cells before in vivo genetic modification (e.g., introduction of the CAR gene) is performed (e.g., in vivo virally mediated genetic modification). Without wishing to be bound by theory, Step (1) is typically important for regeneration of the autologous T cells (exhausted or senescent T cells) taken from the immunocompromised cancer patients. The mitochondria can be co-incubated with the cells ex vivo at ratios between 0.2:1 to 5000:1, for example at ratios of 0.2:1, 0.5:1, 1:1, 10:1, 50:1, 100:1, 200:1, 500:1, 1000:1 or 5000:1.

In order to boost immune cell activity, such as CAR-T cell activity, in vivo, mitochondria can also be delivered (4) along with the immune cells into a patient. In a particular embodiment, mitochondria preparations are delivered in vivo to immune cells (1) before, (2) concurrently with, or (3) after in vivo genetic modification (e.g., introduction of the CAR gene) is performed (e.g., in vivo virally mediated genetic modification). In a particular embodiment, mitochondria preparations are delivered in vivo to immune cells after ex vivo genetic modification (e.g., introduction of the CAR gene) is performed. In a particular embodiment of the present invention, the CAR-T cells or other immune cells are delivered via a systemic (intravenous) infusion while mitochondria are delivered (5) via intratumoral injection, (6) intraorgan injection, (7) intra-tissue injection, or (8) through the organ-specific or tissue-specific vasculature.

### EXAMPLES

The following are examples of methods and compositions of the invention. It is understood that various other embodiments may be practiced, given the general description provided herein.

### Example 1a: Isolating Mitochondria from Tissue Samples or Cultured Cells

Experiments were performed to isolate mitochondria from tissue samples or cultured cells.

### Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
1 M KH2PO4 Stock Solution.
1 M MgCl₂ Stock Solution.
Homogenizing Buffer (pH 7.2): 300 mM sucrose, 10 mM K-HEPES, and 1 mM K-EGTA. Stored at 4° C.
1×PBS (ThermoFisher, 10010031)
1×PBS was prepared by pipetting 100 mL 10×PBS into 1 L double distilled H₂O. Subtilisin A Stock was prepared by weighing out 2 mg of Subtilisin A into a 1.5 mL microfuge tube. Stored at −20° C. until use. Prepared at 2 mg/ml in Homogenizing Buffer.
   Isolation of Mitochondria from Tissue

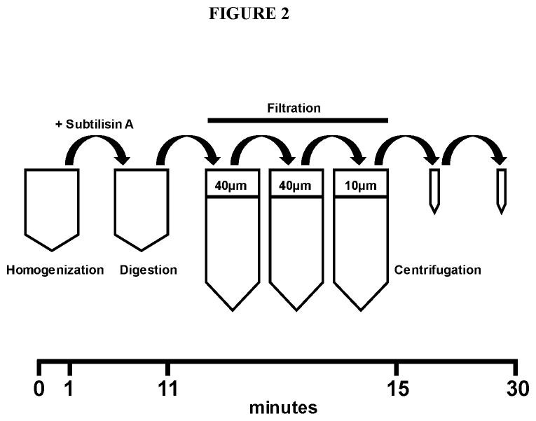

A scheme outlining the procedural steps in the isolation of mitochondria using tissue dissociation and differential filtration is shown in FIG. 2. Two, 6 mm biopsy fresh sample punches taken from the skeletal muscles were transferred to 5 mL of Homogenizing Buffer in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was centrifuged at 750×g for 4 minutes (as an optional step). Afterwards, the homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. The filtrate was re-filtered through a new pre-wetted 6 μm mesh filter in a 50 mL conical centrifuge tube on ice. The resulting filtrate was either used immediately or concentrated by centrifugation. In the case of concentration, the filtrate was transferred to 1.5 mL microfuge tubes and centrifuged at 9000×g for 10 minutes at 4° C. The supernatant was removed, and the pellets containing mitochondria were re-suspended, and combined in 1 mL of homogenizing buffer.

Isolation of Mitochondria from Cultured Cells

Mitochondria were also isolated from the cultured cells, for example, from human cardiac fibroblast (HCF) cell line (obtained from ScienCell Research Laboratories, Carlsbad, CA).

### Culture of the Human Cardiac Fibroblast (HCF) Cells

Human cardiac fibroblasts (HCF) were maintained in Fibroblast Medium-2 containing fetal bovine serum, fibroblast growth supplement-2, and antibiotic (penicillin/streptomycin) solution according to the supplier's directions (ScienCell). The cells were maintained as a monolayer at 37° C. in humidified atmosphere of 5% CO₂ and were passaged when 90% confluence was reached.

### Preparation of the Human Cardiac Fibroblast (HCF) Cells

HCF cells from two flasks (T150) at a confluency of 80% were washed once with PBS. Then trypsin was used to detach the cells according to the supplier instructions (ScienCell Research Laboratories, Carlsbad, CA). The reaction was stopped by adding trypsin neutralizing solution according to the supplier's instructions (ScienCell Research Laboratories, Carlsbad, CA). The cells were collected in a 50 ml centrifuge tube and centrifuged for 5 minutes at 1000 rpm (190×g). The supernatant was discarded and three washes with 1×PBS were performed in total.

Preparation of culture cells different from HCF, should be done according to the manufacturer's instructions. Of note, the cells used as the source of mitochondria can be adherent, semi-adherent or in suspension.

The mitochondria isolation procedure was essentially the same as the procedure for isolating mitochondria from the tissue samples, except that human fibroblast were used rather than biopsy samples.

Alternatively, mitochondria could be isolated by repetitive centrifugation (Kesner et al., 2016, “Characteristics of Mitochondrial Transformation into Human Cells”, Sci Rep 6:26057). In brief, the cells were collected by trypsinization, suspended in PBS, and centrifuged (5 minutes, 250×g) twice. Mitochondrial isolation procedures were performed at 4° C. or on ice. The centrifuged cells were re-suspended in mitochondrial isolation buffer (320 mM sucrose, 5 mM Tris-HCl, pH 7.4, 2 mM EGTA), and homogenized with a Dounce homogenizer. Nuclei and cell debris were removed by two centrifugations at 3000×g for 5 minutes and the supernatant was collected (optional step). The supernatant was then centrifuged at 12,000×g for 10 minutes, and the mitochondrial pellet was re-suspended in mitochondrial isolation buffer. Mitochondrial concentration was determined by Bradford assay.

### Mitochondrial Number

Viable mitochondrial number was determined by labeling an aliquot (10 μL) of isolated mitochondria with MitoTracker Orange CMTMRos (5 μmol/L; Thermo Fisher Scientific). Aliquots of labeled mitochondria were spotted onto slides and counted using a spinning disk confocal microscope with a 63×C-apochromat objective (1.2 W Korr/0.17 NA, Zeiss). Mitochondria were counterstained with the mitochondria-specific dye MitoFluor Green (Thermo Fisher Scientific). Appropriate wavelengths were chosen for measurement of autofluorescence and background fluorescence with use of unstained cells and tissue. Briefly, 1 μL of labeled mitochondria was placed on a microscope slide and covered. Mitochondrial number was determined at low (×10) magnification covering the full specimen area using MetaMorph Imaging Analysis software.

### Example 1b: Isolating Mitochondria from Cultured Cells

Experiments were performed to isolate mitochondria from cultured cells.

### Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
Homogenizing Buffer (pH 7.2): 300 mM sucrose, 10 mM K-HEPES, and 1 mM K-EGTA. Stored at 4° C.
1×PBS (ThermoFisher, 10010031)
Subtilisin A Stock was prepared by weighing out 2 mg of Subtilisin A into a 1.5 mL microfuge tube. Stored at −20° C. until use. Prepared at 2 mg/ml in Homogenizing Buffer.

### Culture of the Human Cardiac Fibroblast (HCF) Cells

Human cardiac fibroblasts (HCF) (obtained from ScienCell Research Laboratories, Carlsbad, CA) were cultured as described in Example 1a and the cells were passaged when 90% confluence was reached.

Preparation of culture cells different from HCF, should be done according to the manufacturer's instructions. Of note, the cells used as the source of mitochondria can be adherent, semi-adherent or in suspension.

Isolation of Mitochondria from Cultured Cells

Mitochondria were also isolated from cultured cells, for example, from human cardiac fibroblast (HCF) cell line. The preparation of HCF cells was done according to the of Example 1a. The HCF cells from each flask were then transferred to 5 mL of Homogenizing Buffer in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. Optionally, the filtrate was re-filtered again through a new pre-wetter 5 μm mesh filter in a 50 mL conical centrifuge tube on ice. The resulting filtrate was either used immediately or concentrated by centrifugation. In the case of concentration, the filtrate was transferred to 1.5 mL microfuge tubes and centrifuged at 9500×g for 5 minutes at 4° C. Three washes were performed at the same centrifugation speed.

### Quantification of Isolated Mitochondria

The isolated mitochondria were suspended in the Homogenizing Buffer of Example 1b and kept on ice until use. Mitochondria quantity, in preparation for varying dosage administration, was measured using a Qubit™ Fluorometer (ThermoFisher Scientific/Invitrogen), employing the Qubit™ Protein Assay kit in accordance with the manufacturer's instructions. For the protein concentration measurement, the mitochondria were resuspended in PBS (ThermoFisher, 10010031). The mitochondria dosage was estimated in terms of protein content expressed in μg.

### Example 2: T Cell Isolation, Activation and Culture

CD8⁺ T cells were isolated from buffy coats of healthy donors. Peripheral blood mononuclear cells (PBMC) were collected by density gradient centrifugation using Ficoll Paque plus (Cytiva, 17144002) according to the manufacturer's instructions. Human CD8⁺ T cells were harvested from the PBMCs using the EasySep™ Human CD8⁺ T Cell Isolation Kit (Stemcell, 17953) and The Big Easy” EasySep™ Magnet (Stemcell, 18001). Isolated CD8⁺ T cells were activated with Dynabeads Human T-Activator CD3/CD28 (ThermoFisher, 111.32D), in a 1 to 1 ratio, in presence of 100 U/ml of recombinant human IL-2 (Peprotech, 200-02). CD8⁺ T cells were cultured in RPMI 1640 medium GlutaMAX™ Supplement 500 ml (ThermoFisher, 61870010), supplemented with 1% L-glutamine (ThermomFisher, 25030024), 1% penicillin-streptomycin (10′000 U/mL, Gibco, 15140122), 1% non-essential amino acid (NEAA, ThermoFisher, 11140050), 1% sodium pyruvate (ThermoFisher, 11360070), 10% fetal bovine serum and 0.1% 2β-mercaptoethanol (Gibco, 31350-010). CD8⁺ T cells were plated at 0.5 Million of cells/mL and split when the cells reached a confluency of 2 Million cells/mL or when the medium was turning yellow.

### Example 3: T Cell Transplantation

CD8⁺ T cells were plated at 0.5 Million cells/mL in a 24 well plate 24 h prior to mitochondria transplantation. When the mitochondria were isolated, CD8⁺ T cells were collected and centrifuged for 5 minutes at 1500 rpm (430×g). The supernatant was discarded and the cells were resuspended in fresh T cell medium at the concentration of 1 Million cell 100 μL. The T cell medium is described under Example 2.

Transplanted CD8⁺ T cells were incubated for 4 h with isolated mitochondria in a range of 10 μg to 100 μg of protein per 1 Million of CD8⁺ T cells in a final volume of 200 μL of T cell medium in each well of the 24 well plate. 4 h post co-incubation of exogenous mitochondria and CD8⁺ T cells, 1.8 ml of fresh T cell medium was added per well.

### Example 4: Mitochondria Labeling and Internalization

### Example 4.1—T Cell Transplantation with Stained Isolated Mitochondria

CD8⁺ T cells are plated at 0.5 Million cells/mL in a 24 well plate 24 h prior to mitochondria transplantation. Mitochondria are isolated according to procedure described under Example 1b. Mitochondria are then stained for 10 to 15 minutes at 37° C. with Mitotracker Red CMXRos (ThermoFisher, M7512) and Mitotracker Green FM (ThermoFisher, M7514) at 200 nM in the Homogenizing Buffer of Examples 1b. Three washes of the stained mitochondria are performed with Homogenizing Buffer of Examples 1b at 9500×g for 5 minutes at 4° C. and the supernatant of the last wash is saved as a control. CD8⁺ T cells are collected and centrifuged for 5 minutes at 1500 rpm (430×g). The supernatant is removed, and the cells are resuspended in fresh T cell medium at 1 Million cells/100 μL. The T cell medium is described under Example 2. Stained mitochondria are (immediately) added to the T cells to obtain a final volume of 200 μL per well of a 24 well plate. The last wash of the stained mitochondria is added in an equivalent volume to the control non-transplanted CD8⁺ T cells. The integration of the stained mitochondria is evaluated by flow cytometry (e.g., data acquired with FACSLyric (BD Biosciences)) or by fluorescence microscopy (Keyence microscope, BZ-X810) from 5 minutes to 24 h post transplantation. In case of a co-incubation of exogenous mitochondria and CD8⁺ T cells longer than 4 h, 1.8 ml of fresh T cell medium is added per well.

### Example 4.2—Staining Post Mitochondria Transplantation

Transplanted CD8⁺ T cells are incubated for 4 h with isolated mitochondria in a range of 10 μg to 100 μg of protein per 1 Million of CD8⁺ T cells in a final volume of 200 μL of T cell medium in each well of the 24 well plate. 4 h post co-incubation of exogenous mitochondria and CD8*T cells, 1.8 ml of fresh T cell medium is added per well. Mitochondrial respiration and mass are evaluated in transplanted cells 24 h post co-incubation. The dyes Mitotracker Red CMXRos (ThermoFisher, M7512) and Mitotracker Green FM (ThermoFisher, M7514) are diluted to a final concentration of 100 nM in RPMI 1640 medium, no phenol red (ThermoFisher, 11835030), supplemented with 1% penicillin-streptomycin (10′000 U/mL, Gibco, 15140122), 5% fetal bovine serum. 100 μl of the staining is added per 1 Million of CD8⁺ T cells and the staining is performed for 15 minutes at 37° C. The cells are then washed twice with FACS buffer (lx PBS (ThermoFisher, 10010031), 2% FBS, 1% EDTA 0.5M (Sigma-Aldrich, E6758)) at 1500 rpm (430×g) for 5 minutes. The supernatant is discarded and the CD8⁺ T cells are resuspended in 300 μL of FACS buffer and acquired on a FACS machine (FACSLyric, BD Biosciences).

### Example 5: Increased Proportion of Memory CD8⁺ T Cells In Vitro Upon Mitochondria Transplantation

The proportion of memory T cells was evaluated by flow cytometry at day 9 post exogenous mitochondria transplantation into CD8⁺ T cells isolated from healthy donors and subsequently cultivated.

### Procedure

(i) T cell isolation, activation, and culture was performed as described in Example 2.
(ii) Mitochondria isolation: Mitochondria were isolated from human cardiac fibroblasts (HCF) as previously described in Example 1b. The isolated mitochondria were suspended in the Homogenizing Buffer of Example 1b and kept on ice until use. Mitochondria quantity, in preparation for varying dosage administration, was measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in μg.
(iii) Day 9 post transplantation, the staining was performed on ice according to the manufacturer's instructions using anti-human CD45RA APC (Biolegend, 304112), anti-human CD45RO PB (Biolegend, 304223) and anti-human CD62L FITC (Biolegend, 304804). Depending on the surface expression, the CD8⁺ T cells were classified as naïve (CD62L+, CD45RA+, CD45RO−), stem cell-like memory (CD62L+, CD45RA+, CD45RO+), central memory (CD62L+, CD45RA−, CD45RO+), effector memory (CD62L−, CD45RA−, CD45RO+) or effector (CD62L−, CD45RA+, CD45RO−). The portion of the different subsets is compared between untreated and CD8⁺ T cells transplanted with exogenous mitochondria.

### Results

### Increased Proportion of Central and Effector Memory CD8+ T Cells Day 9 Post Mitochondria Transplantation

To investigate the ability of transplanted mitochondria to favor the survival and/or differentiation and/or selection of memory CD8⁺ T cells from a bulk population, mitochondria were transplanted into CD8⁺ T cells at dosage levels of 30 μg and 100 μg of mitochondria per 1 million CD8⁺ T cells day 12 post activation. On day 9 post transplantation, CD8⁺ T cells were stained, analyzed by flow cytometry using a FACSLyric (BD Biosciences) and classified as naïve (CD62L+, CD45RA+, CD45RO−), stem cell-like memory (CD62L+, CD45RA+, CD45RO+), central memory (CD62L+, CD45RA−, CD45RO+), effector memory (CD62L−, CD45RA−, CD45RO+) or effector (CD62L−, CD45RA+, CD45RO−).

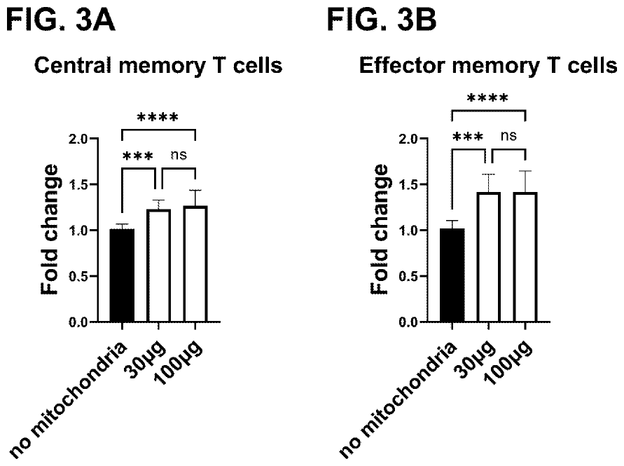

As shown in FIG. 3, there is a clear increased proportion of central and effector memory CD8⁺ T cells upon mitochondria transplantation compared to untreated CD8⁺ T cells. A significant enhancement of central and effector memory CD8⁺ T cells was detected with a dosage of 30 μg and 100 μg of mitochondria.

### Example 6: Increased Proportion of Memory CD8+ T Cells In Vivo Upon Mitochondria Transplantation

The proportion of effector and memory T cells is evaluated over time in a mounted immune response against an acute infection. CD45.1 mouse OT-I T cells restricted against ovalbumin (OVA) peptide are activated and transplanted with exogenous mitochondria, followed by injection into CD45.2 C57/B6 mice subsequently infected with Listeria-OVA. The treated group is compared to the mounted immune response of OT-I T cells not transplanted with mitochondria.

### Procedure

(i) Mouse CD8⁺ T cell isolation is performed according to EasySep™ mouse CD8⁺ T Cell Isolation Kit (StemCell, Cat. #19853). T cell activation and expansion is performed by using CD3/CD28 Dynabeads (Gibco, Cat. #11456.D) at a ratio 1-1 and recombinant IL-2 (50 U/ml). CD8⁺ T cells are plated at 0.5 Million of cells/mL and split when the cells reach a confluency of 2 Million cells/mL or when the medium is turning yellow.
(ii) Mitochondria isolation of an OT-I mouse: Mitochondria are isolated from skeletal muscle as previously described in Example 1a. The isolated mitochondria are suspended in the Homogenizing Buffer of Example 1a and kept on ice until use. Mitochondria quantity, in preparation for varying dosage administration, is measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in μg.
(iii) Mouse CD8⁺ T cell are transplanted as previously described in Example 3 at day 7 post activation.
(iv) Mice (CD45.2) are injected with 20′000 OT-I CD8⁺ T cells (CD45.1) transplanted 24 h before and are subsequently infected with 2′000 colony forming units (cfu) of Listeria-OVA. The persistence and memory differentiation of CD8⁺ T cells are evaluated in the blood of animals over time (day 7, day 14, day 21) and organs (spleen, lymph nodes (LNs) at day 21). Staining from the blood or processed organs: LIVE/DEAD Fixable dye Aqua Dead (ThermoFisher, L34957), anti-mouse CD8α Pe/Texas Red (Abcam ab25294), anti-mouse CD45.1 BV650 (BD 563754), anti-mouse CD45.2 BV421 (BD 562895), anti-mouse KLRG1 PE-Cy7 (BioLegend 138415), anti-mouse CD127 PE (BioLegend 121111), anti-mouse CD44 APC-Cy7 (BD 560568), anti-mouse CD62L PerCP/Cyanine5.5 (BioLegend 104431). The mounted immune response of the OT-I T cells against Listeria-OVA is classified as short-lived effector cells (SLECs) (KLRG1+ CD127− and/or CD44+ CD62L−) and memory precursor cells (MPECs) (KLRG1− CD127+ and/or CD44+ CD62L+).
(v) The cytokine production is assessed on the day of sacrifice, 4 h post peptide restimulation (OVA peptide). Cells are collected from homogenized spleen and LNs are plated in a 96 well plate. The cells are incubated with 10 μM of SIINFEKL (OVA) peptide or PMA/ionomycin for 30 min, followed by another 4 h of restimulation in the presence of Golgistop (BD) and Golgiplug (BD). The cells are collected, fixed and permeabilized for intracellular cytokine staining: anti-mouse IFNγ PerCP/Cyanine5.5 (BioLegend 505821), anti-mouse TNFα Pacific Blue (BioLegend 506318), anti-mouse IL-2 PE (BioLegend 503807) and anti-mouse Granzyme B FITC (BioLegend 515403) production are assessed.

### Results

### Exogenous Mitochondria Transplantation Promotes Memory Cell Formation and Persistence During a Mounted Immune Response

Over time, the proportion of mouse short-lived effector cells (SLECs) (KLRG1+ CD127− and/or CD44+ CD62L−) is reduced and memory precursor cells (MPECs) (KLRG1− CD127+ and/or CD44+ CD62L+) is increased in mice injected with transplanted OT-I CD8⁺ T cells. Upon peptide restimulation, the cytokine production of OT-I CD8⁺ T cells in the treated group with exogenous mitochondria is higher compared to untreated group.

### Example 7: Increased Proportion of Memory-Like CD8⁺ T Cells from TILs In Vitro Upon Mitochondria Transplantation

The proportion of memory-like T cells is evaluated by flow cytometry over time post exogenous mitochondria transplantation into cultivated human TILs.

### Procedure

(i) TIL isolation and culture: Surgically resected tumor mass is digested using enzymes such as collagenase type IV (Sigma Aldrich) and Pulmozyme (Roche) generating a single cell suspension. TIL are expanded with high dose of IL-2 as previously described (van den Berg J H, et al. J Immunother Cancer 2020; 8:e000848. doi:10.1136/jitc-2020-000848). If the proportion of TIL CD8⁺ T cells is sufficient among the bulk TIL population, isolation of CD8⁺ T cells is performed as described in Example 2
(ii) Mitochondria isolation: Mitochondria are isolated from human cardiac fibroblasts (HCF) as previously described in Example 1b. The isolated mitochondria are suspended in the Homogenizing Buffer of Example 1b and kept on ice until use. Mitochondria quantity, in preparation for varying dosage administration, is measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in μg.
(iii) Over time in a range from 4 h to two weeks post transplantation, the staining is performed on ice according to the manufacturer's instructions using anti-human CD45RA APC (Biolegend, 304112), anti-human CD45RO PB (Biolegend, 304223) and anti-human CD62L FITC (Biolegend, 304804). Depending on the surface expression, the CD8⁺ T cells are classified as naïve (CD62L+, CD45RA+, CD45RO), stem cell-like memory (CD62L+, CD45RA+, CD45RO+), central memory (CD62L+, CD45RA−, CD45RO+), effector memory (CD62L−, CD45RA−, CD45RO+) or effector (CD62L−, CD45RA+, CD45RO−). The portion of the different subsets is compared between control and TIL CD8⁺ T cells transplanted with exogenous mitochondria.

### Results

### Increased Proportion of Memory-Like TIL CD8+ T Cells Post Mitochondria Transplantation

Transplantation of exogenous mitochondria into TILs from a bulk population promotes the survival and the selection of memory-like TILs.

### Example 8: Adoptive Cell Transfer of Transplanted TILs Rechallenged in Tumor-Bearing Mice or Upon Acute Infection Display an Enhanced Recall Response

OT-I TILs extracted and isolated from an OVA-expressing tumor are transplanted with exogenous mitochondria. To evaluate the properties of the selected memory-like TILs post in vitro culture, treated or untreated TILs are adoptively transferred and rechallenged into tumor-bearing mice or upon acute infection. In OVA-restricted tumor-bearing mice, the recall capacity of OT-I TILs is assessed over time by measuring tumor growth, mice survival and persistence of the transferred cells infiltrating the cancer mass and in lymphoid organs. In an acute infection setting, OT-I TILs are adoptively transferred in animal subsequently infected with an OVA-expressing virus or bacteria. The mounted immune response is evaluated over time in the blood and lymphoid organs between transplanted TILs or untreated TILs.

### Procedure

(i) Mouse TIL generation and extraction: CD45.2 C57/B6 mice are engrafted subcutaneously with 200′000 OVA-expressing tumor cells on one flank. 6 days post engraftment, 100′000 CD45.1 OT-I T cells are adoptively transferred intravenously. 21 days post engraftment and/or when an appropriate tumor size is reached, tumors are harvested and dissociated with the Tumor Dissociation Kit (130-096-730, Miltenyi Biotec) following manufacturer's instructions. To select mouse CD8⁺ T cell from the tumors, isolation is performed according to EasySep™ mouse CD8⁺ T Cell Isolation Kit (StemCell, Cat. #19853). To further select OT-I TILs, FACS-based cell sorting is performed according to LIVE/DEAD−, CD45.1+, CD8+.
(ii) Mitochondria isolation from mouse OT-I: Mitochondria are isolated from skeletal muscle as previously described in Example 1a. The isolated mitochondria are suspended in the Homogenizing Buffer of Example 1a and kept on ice until use. Mitochondria quantity, in preparation for varying dosage administration, is measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in μg.
(iii) Mouse CD8⁺ T cell are transplanted as previously described in Example 3.
(iv) Rechallenge in tumor-bearing mice: CD45.2 C57/B6 mice are engrafted subcutaneously with 200′000 OVA-expressing tumor cells on one flank. 5 days post engraftment, 5Gy whole body radiation is applied. 6 days post engraftment, 10′000 transplanted CD45.1 OT-I TILs are adoptively transferred intravenously. Tumor growth is measured every 2-3 days using a caliper. 21 days post engraftment and/or when an appropriate tumor size is reached, tumors are harvested and dissociated with the Tumor Dissociation Kit (130-096-730, Miltenyi Biotec) following manufacturer's instructions. Infiltration at the tumor and persistence in lymphoid organs is assessed by flow cytometry by staining: LIVE/DEAD Fixable dye Aqua Dead (ThermoFisher, L34957), anti-mouse CD8α Pe/Texas Red (Abcam ab25294), anti-mouse CD45.1 BV650 (BD 563754), anti-mouse CD45.2 BV421 (BD 562895), anti-mouse KLRG1 PE-Cy7 (BioLegend 138415), anti-mouse CD127 PE (BioLegend 121111), anti-mouse CD44 APC-Cy7 (BD 560568), anti-mouse CD62L PerCP/Cyanine5.5 (BioLegend 104431).
(v) Rechallenge upon an acute infection: Mice (CD45.2) are injected with 10′000 OT-I TILs (CD45.1) one day post transplantation and are subsequently infected with 2′000 cfu of Listeria-OVA. The persistence and memory differentiation of OT-I TILs are evaluated in the blood of animals over time (day 7, day 14, day 21) and organs (spleen, LNs at day 21). Staining from the blood or processed organs: LIVE/DEAD Fixable dye Aqua Dead (ThermoFisher, L34957), anti-mouse CD8α Pe/Texas Red (Abcam ab25294), anti-mouse CD45.1 BV650 (BD 563754), anti-mouse CD45.2 BV421 (BD 562895), anti-mouse KLRG1 PE-Cy7 (BioLegend 138415), anti-mouse CD127 PE (BioLegend 121111), anti-mouse CD44 APC-Cy7 (BD 560568), anti-mouse CD62L PerCP/Cyanine5.5 (BioLegend 104431). The recall immune response of the OT-I TILs against Listeria-OVA is classified as short-lived effector cells (SLECs) (KLRG1+CD127− and/or CD44+ CD62L−) and memory precursor cells (MPECs) (KLRG1− CD127+ and/or CD44+ CD62L+).

### Results

### Transplanted TILs Display an Improved Recall Capacity in Tumor-Bearing Mice and Upon an Acute Infection.

Transplanted TILs rechallenged in tumor-bearing mice or in infected mice, display hallmark of memory cells, as shown by enhanced persistence, improved recall capacity and better tumor control in tumor-bearing animal.

### Example 9: Transplanted CD8⁺ T Cells have an Enhanced Capacity to Compete for Survival Signals

To evaluate the capacity of transplanted T cells to compete efficiently for survival signal, a co-transfer of treated and untreated cells is performed within the same host. CD45.1 mouse OT-I T cells restricted against ovalbumin (OVA) peptide are activated and transplanted with exogenous mitochondria whereas CD45.1.2 OT-I T cells are not transplanted. CD45.1 treated OT-I and CD45.1.2 untreated OT-I are co-transferred into CD45.2 C57/B6 mice subsequently infected with Listeria-OVA. The treated group is compared to the mounted immune response of OT-I T cells not transplanted with mitochondria within the same host and competing for limited survival signals.

### Procedure

(i) Mouse CD8⁺ T cell isolation is performed according to EasySep™ mouse CD8*T Cell Isolation Kit (StemCell, Cat. #19853). T cell activation and expansion is performed by using CD3/CD28 Dynabeads (Gibco, Cat. #11456.D) at a ratio 1-1 and recombinant IL-2 (50 U/ml). CD8⁺ T cells are plated at 0.5 Million of cells/mL and split when the cells reach a confluency of 2 Million cells/mL or when the medium is turning yellow.
(ii) Mitochondria isolation of an OT-I mouse: Mitochondria are isolated from skeletal muscle as previously described in Example 1a. The isolated mitochondria are suspended in the Homogenizing Buffer of Example 1a and kept on ice until use. Mitochondria quantity, in preparation for varying dosage administration, is measured using a Qubit™ Fluorometer (ThermoFisher Scientific/Invitrogen), employing the Qubit™ Protein Assay Kit in accordance with the manufacturer's instructions. The mitochondria dosage is estimated in terms of protein content expressed in μg.
(iii) Mouse CD8⁺ T cell are transplanted as previously described in Example 3 at day 7 post activation.
(iv) Mice (CD45.2) are injected with 10′000 OT-I CD8⁺ T cells (CD45.1) one day post transplantation and with 10′000 OT-I CD8⁺ T cells (CD45.1.2) untreated. The mice are subsequently infected with 2′000 cfu of Listeria-OVA. The persistence and memory differentiation of CD8⁺ T cells are evaluated in the blood of animals over time (day 7, day 14, day 21) and organs (spleen, LNs at day 21). Staining from the blood or processed organs: LIVE/DEAD Fixable dye Aqua Dead (ThermoFisher, L34957), anti-mouse CD8α Pe/Texas Red (Abcam ab25294), anti-mouse CD45.1 BV650 (BD 563754), anti-mouse CD45.2 BV421 (BD 562895), anti-mouse KLRG1 PE-Cy7 (BioLegend 138415), anti-mouse CD127 PE (BioLegend 121111), anti-mouse CD44 APC-Cy7 (BD 560568), anti-mouse CD62L PerCP/Cyanine5.5 (BioLegend 104431). The mounted immune response of the OT-I T cells against Listeria-OVA is classified as short-lived effector cells (SLECs) (KLRG1+ CD127− and/or CD44+ CD62L−) and memory precursor cells (MPECs) (KLRG1− CD127+ and/or CD44+ CD62L+).

### Results

### Enhanced Capacity of Transplanted Cells to Compete for Limited Survival Signals

OT-I T cells transplanted with exogenous mitochondria compete better for the limited survival signals post acute infection. Consequently, the proportion of treated T cells circulating in the blood and lymphoid organs is enhanced compared to untreated T cells.

### Example 10: Mitochondrial Transfer Increases Persistence of CAR-T Cells In Vivo

Bulk CD8 T cells from healthy donor are transplanted with exogenous mitochondria and cultured to select central memory an effector memory T cell over time. CD8 T cells from healthy donor are transduced to express anti-CD19 CAR-T constructs (anti-CD19scFv-FLAG-CD28-CD3ζ, Promab). The mounted immune response of CAR-T cells treated with mitochondria or not are evaluated in mouse xenograft models of B-cell lymphoma.

### Procedure

(i) CAR-T cell culture is performed as described in Example 2.
(ii) Mitochondria isolation: Mitochondria are isolated from Human Cardiac Fibroblast (HCF) according to the procedure described in Example 1b.
(iii) Quantification of isolated mitochondria: The mitochondria dosage is estimated in terms of protein content expressed in μg, according to the procedure described in Example 1b.
(iv) CAR-T cell transplantation according to the procedure of Example 3. An amount of 30 μg or 100 μg of mitochondria is transplanted in the CAR-T cells.

### Lymphoma Model

Nine-week-old female NOD/SCID mice (non-obese diabetic; deficient for T cells, macrophages and NK cells; Taconic, Denmark) are subcutaneously (s.c.) injected with human Burkitt's lymphoma CD19⁺ Raji cells (2.5×10⁶ cells/mouse). Animals are randomized into treatment groups when the tumors reached the size of 60-100 mm³; 5-8 mice per group with equal tumor size are selected for the treatment. The animals received i.v. injections of 10⁷ mock-transduced T cells, or anti-CD19 CAR-T cells, or mitochondria-enhanced anti-CD19 CAR-T cells. Tumor size is measured in two dimensions with a caliper-like instrument. Individual tumor volumes (V) are calculated by the formula V=0.56×(length+width)². Upon reaching the humane endpoint with a tumor volume of 1,500 mm³, the animals are sacrificed by cervical dislocation. The Kaplan-Meier survival plots are generated using the software program PRISM (GraphPad) and the survival curves are compared using a log-rank (Mantel-Cox) test.

### Leukemia Model

Eight-week-old male NSG (NOD/SCID gamma mouse; deficient for T cells, B cells and NK cells) mice purchased from Jackson Laboratories are housed in the vivarium in sterile cages. Raji/Luc-GFP cells (10⁶) in 100 μL PBS are injected i.v. via the lateral tail vein using an insulin syringe (designated as day 0). Luciferase activity is measured on day 6 via bioluminescence imaging to assess tumor burden. On day 7, 10⁷ mock-transduced T cells, anti-CD19 CAR-T cells, or mitochondria-enhanced anti-CD19 CAR-T cells are prepared in 100 μL PBS, and injected i.v. using an insulin syringe. Tumor progression is monitored by bioluminescence imaging using an IVIS imaging system. At day 60, surviving mice are euthanized, spleen and bone marrow cells harvested and re-suspended in a total volume of 2 mL of flow cytometry (FACS) buffer (PBS, supplemented with 2% FCS). Two hundred microliters of the cell suspension are then labeled with anti-human CD3 PE and anti-human CD45 APC antibodies, and analyzed by flow cytometry to determine the percentage of human T cells.

Relative to non-enhanced or control CAR-T cells, the CAR-T cells enhanced with exogenous mitochondria demonstrate higher anti-tumor activity (longer median survival) in the treated mice.

### Example 11: Impact of Mitochondria Transplantation on Treg Survival and Selection In Vitro

The proportion of Tregs is evaluated by flow cytometry over time post exogenous mitochondria transplantation into CD4⁺ T cells bulk population isolated from healthy donors and subsequently cultivated.

### Procedure

(i) CD4⁺ T cell isolation, activation and culture: CD4⁺ T cells are isolated from buffy coats of healthy donors. Peripheral blood mononuclear cells (PBMC) are collected by density gradient centrifugation using Ficoll Paque plus (cytiva, 17144002) according to the manufacturer's instructions. Human CD4⁺ T cells are harvested from the PBMCs using the EasySep™ Human CD4⁺ T Cell Isolation Kit (Stemcell, 17952) and The Big Easy” EasySep™ Magnet (Stemcell, 18001). Isolated CD4⁺ T cells are activated with Dynabeads Human T-Activator CD3/CD28 (ThermoFisher, 111.32D), in a 1-1 ratio, in presence of 100 U/ml of recombinant human IL-2 (Peprotech, 200-02). CD4⁺ T cells are cultured in RPMI 1640 medium GlutaMAX™ Supplement (ThermoFisher, 61870010), supplemented with 1% L-glutamine (ThermomFisher, 25030024), 1% penicillin-streptomycin (10′000 U/ml, Gibco, 15140122), 1% non-essential amino acid (NEAA, ThermoFisher, 11140050), 1% sodium pyruvate (ThermoFisher, 11360070), 10% fetal bovine serum and 0.1% 2β-mercaptoethanol (Gibco, 31350-010). CD4⁺ T cells are plated at 0.5 mio of cells/ml and are split when the cells reach a confluency of 2mio cells/ml or when the medium is turning yellow.
(ii) CD4⁺ T cells transplantation: CD4⁺ T cells are transplanted between day 1 to day 20 post activation, with various doses of mitochondria in a range of 10 μg to 100 μg per million of CD4⁺ T cells.
(iii) Staining post transplantation of CD4⁺ T cells: in a range of 1 day to 20 days, CD4⁺ T cells are stained and analyzed by Flow cytometry at various time points. The staining is performed on ice according to the manufacturer's instructions using anti-human CD45RA APC (BioLegend, 304112), anti-human CD45RO PB (BioLegend, 304223), anti-human CD25 FITC (BioLegend, 302604) and anti-human CD127 Pe (BioLegend, 351304). Depending on the surface expression of the mentioned markers, CD4⁺ T cells are classified as naïve (CD25−, CD127+, CD45RA+, CD45RO−), Treg (CD25+, CD127−, CD45RA+, CD45RO−), central memory (CD25+, CD127+, CD45RA−, CD45RO+), effector memory (CD25−, CD127+, CD45RA−, CD45RO+) or effector (CD25+, CD127−, CD45RA+/−, CD45RO+/−). The portion of the different subsets is compared between control and CD4⁺ T cells transplanted with exogenous mitochondria. In addition, the level of FOXP3 in the Treg population is assessed post mitochondria transplantation using True-Nuclear Human Treg Flow Kit (BioLegend, 320027) according to the manufacturer's instructions.

### Results

Exogenous Mitochondria Transplantation Promotes Treg Selection from CD4⁺ T Cell Bulk Population

Upon mitochondria transplantation, Treg are found in a higher proportion in the bulk population compared to CD4⁺ T cells that are not treated with exogenous mitochondria. This selection method can be used to increase the proportion of Tregs from a bulk population of CD4⁺ T cells for adoptive cell therapy treating autoimmune diseases.

### Incorporation by Reference

The entire disclosures of all patent and non-patent publications cited herein are each incorporated by reference in their entireties for all purposes.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and sub-combinations of the various elements, features, functions, and/or properties disclosed herein. The following claims particularly point out certain combinations and sub-combinations regarded as novel and nonobvious. Inventions embodied in other combinations and sub-combinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed:

1. Immune cells, such as human immune cells, treated with isolated viable mitochondria in an amount effective to:
(i) enhance the survival of adaptive immune cells, (ii) promote the selection of adaptive immune cells, or (iii) a combination thereof, relative to immune cells, e.g. immune cells, not treated with isolated viable mitochondria.

2. Immune cells, such as human immune cells, comprising exogenous mitochondria, such as exogenous isolated viable mitochondria, in an amount effective to:
(i) enhance the survival of adaptive immune cells, (ii) promote the selection of adaptive immune cells, or (iii) a combination thereof, relative to immune cells, e.g. human adaptive immune cells, not comprising exogenous isolated viable mitochondria.

3. The immune cells of claim 1 or 2, wherein the immune cells are lymphocytes, such as B cells or T cells, preferably T cells, such as CD8 immune T cells or CD4 immune T cells.

4. The immune cells of claims 1 or 2, wherein the immune cells comprise a chimeric antigen receptor (“CAR”) or an artificial T-Cell Receptor (“TCR”) subunit or combination thereof.

5. The immune cells of claims 1 or 2, wherein the immune cells are produced in vitro or ex vivo.

6. The adaptive immune cells of claim 1 or 2, wherein the adaptive immune cells are memory T cells, such as human memory T cells.

7. The adaptive immune cells of claim 1 or 2, wherein the adaptive immune cells are effector cells, such as effector CD8 T cells or effector CD4 T cells, preferably human effector CD4 T cells or CD8 T cells.

8. The adaptive immune cells of claim 1 or 2, wherein the adaptive immune cells are stem cell-like memory cells or memory-like cells, such as CD8 memory-like cells.

9. The adaptive immune cells of claim 1 or 2, wherein the adaptive immune cells are naïve cells.

10. The adaptive immune cells of claim 1 or 2, wherein the adaptive immune cells are tissue resident memory cells (Trm cells).

11. The adaptive immune cells of any one of claims 1, 2, or 6, wherein the adaptive immune cells are memory CD8 T cells.

12. The adaptive immune cells of any one of claims 1, 2, 6 or 11, wherein the adaptive immune cells are effector memory CD8 T cells, central memory CD8 T cells, or a combination thereof.

13. The adaptive immune cells of claim 1 or 2, wherein the adaptive immune cells are regulatory (Treg) T cells, such as Treg CD4 T cells.

14. A population comprising the immune cells, such as human immune cells, e.g. human adaptive immune cells, according to any one of the preceding claims.

15. The immune cells of any one of the preceding claims, wherein the mitochondria are derived from eukaryotic cell mitochondria.

16. The immune cells of any one of the preceding, wherein the mitochondria are derived from a human cell line.

17. The immune cells of any one of the preceding claims, wherein the mitochondria are derived from a healthy volunteer.

18. The immune cells of any one of the preceding claims, wherein the mitochondria are derived from a patient, such as a cancer patient.

19. The immune cells of any one of the preceding claims, wherein the mitochondria are derived from a patient, such as a patient suffering from an autoimmune disease.

20. The immune cells of any one of the preceding claims, wherein the mitochondria are autologous or allogeneic.

21. The immune cells of any one of the preceding claims, wherein the mitochondria are genetically engineered mitochondria, or mitochondria encapsulated by a liposome or coupled to specific agents.

22. The immune cells of any one of the preceding claims, wherein the effective amount of mitochondria is between 0.0001 ng and 2.5 ng, e.g. between 0.001 ng and 2.0 ng.

23. A composition comprising the immune cells, e.g. adaptive immune cells, or a population of immune cells according to any one of the preceding claims and at least a pharmaceutically acceptable carrier.

24. The composition of claim 22, wherein the pharmaceutically acceptable carrier is formulated for delivery into a human immune cell.

25. The compositions according to claim 23 or 24, wherein the composition is formulated in solid or liquid form.

26. A method of (i) enhancing the survival of immune cells, (ii) promoting the selection of immune cells, or a combination thereof, according to any one of the preceding claims, comprising the step of:
(a) activating the immune cells in vitro in a cell-free medium with specific activating receptor agonist antibodies capable of driving the adaptive cells (such as T cells) activation; and
(b) exposing the immune cells to a pharmaceutical composition comprising isolated viable mitochondria for at least 3 days, such as for at least 5 days.

27. A method of (i) enhancing the survival of immune cells, (ii) promoting the selection of immune cells, or a combination thereof, according to any one of the preceding claims, comprising the step of:
(a) activating the immune cells in vitro in a cell-free medium with coated CD3/CD28 beads, optionally in presence of recombinant interleukins, such IL-2; and
(b) exposing the immune cells to a pharmaceutical composition comprising isolated viable mitochondria for at least 3 days, such as for at least 5 days.

28. The immune cells, e.g. human immune cells, such as human T cells according to any one of the preceding claims for use in a method of treating a subject in need thereof comprising administering to the subject the immune cells, or population of immune cells of any one of the preceding claims.

29. The immune cells, e.g. human immune cells, such as human T cells according to any one of the preceding claims for use in a method of treating cancer, infectious, inflammatory or autoimmune disease.
